(19) United States
(12) Patent Application Publication
(10) Pub. No.: US 2014/0037748 A1
(43) Pub. Date: Feb. 6, 2014

(54) REDOX RESPONSIVE POLYMERIC NANOCAPSULES FOR PROTEIN DELIVERY

(71) Applicants: Yi Tang, San Gabriel, CA (US); Zhen Gu, Los Angeles, CA (US); Muxun Zhao, Los Angeles, CA (US)

(72) Inventors: Yi Tang, San Gabriel, CA (US); Zhen Gu, Los Angeles, CA (US); Muxun Zhao, Los Angeles, CA (US)

(73) Assignee: The Regents of the University of California, Oakland, CA (US)

(21) Appl. No.: 14/110,058

(22) PCT Filed: Apr. 13, 2012
(86) PCT No.: PCT/US12/33515

§ 371 Date: Oct. 4, 2013

Related U.S. Application Data
(60) Provisional application No. 61/476,094, filed on Apr. 15, 2011.

(51) Int. Cl. A61K 9/51; A61K 38/17; A61K 38/48

(52) U.S. Cl. CPC A61K 9/51; A61K 38/4873; A61K 38/1761
USPC 424/497; 424/490; 424/94.65; 514/18.9

(57) ABSTRACT

The invention provides methods of making and using compositions comprising a polymer shell designed to deliver polypeptides to selected environments. In embodiments of the invention, different environmental conditions are harnessed to allow the selective degradation of the polymer shell and the consequential release of one or polypeptides encapsulated therein. In illustrative embodiments, polymer components of the shell are interconnected by disulfide-containing crosslinker moieties, linkages which maintain the integrity of the polymer shell under certain environmental conditions including those occuring outside of cells, but degrade in an intracellular environment.

### REFERENCE TO RELATED APPLICATIONS

This application claims the benefit under 35 U.S.C. Section 119(e) of copending U.S. Provisional Patent Application Ser. No. 61/476,094, filed on Apr. 15, 2011, entitled “REDOX RESPONSIVE POLYMERIC NANOCAPSULES FOR PROTEIN DELIVERY”, the contents of which are incorporated herein by reference.

### STATEMENT OF GOVERNMENT SUPPORT

This invention was made with Government support under Grant No. HDTRA1-09-1-0001, awarded by the U.S. Department of Defense, Defense Threat Reduction Agency. The Government has certain rights in this invention.

### FIELD OF THE INVENTION

This disclosure generally relates to nanocapsules containing polypeptides. Methods of preparing and using such nanocapsules are also disclosed.

### BACKGROUND OF THE INVENTION

Protein therapeutics that function intracellularly have enormous potential for the treatment of human diseases—especially those caused by the temporary or permanent loss of protein function. For example, many cancer cells do not undergo programmed cell death because proteins in the apoptosis machinery are defective and/or attenuated in expression (see, e.g. Cotter T G. Nat Rev Cancer 2009, 9:501-7). Direct protein delivery to the cytosol of cells can therefore be used to restore or replenish the polypeptide functions of interest and lead to desired cell phenotypes. In addition, the introduction of recombinant proteins designed to regulate transcription can exert artificial control of gene expression levels and lead to reprogramming of cell fate (see, e.g. Zhou H, et al. Cell Stem Cell 2009, 4:381-4). Moreover, in comparison to gene therapy, which is currently the predominant choice of delivery for promising protein therapeutics, direct protein delivery can bypass the requirement of permanent or unintended changes to the genetic makeup of the cell, and is therefore a safer therapeutic alternative (see, e.g. Ford K G, et al. Gene Ther 2001, 8:1-4).

Unfortunately, the development of intracellular protein therapeutics has been hampered by limitations arising from the nature of proteins. These limitations include structural fragility, low serum stability and poor membrane permeability for most proteins that are negatively charged at pH 7. See, e.g. Gupta B, et al. Adv Drug Deliv Rev 2005, 57:637-51; Hirakura T, et al. J Control Release 2010, 142:483-9; Frokjaer S, et al. Nat Rev Drug Discov 2005, 4:298-306; Murthy N, et al. Bioconjug Chem 2003, 14:412-9; Haag R, et al. Angew Chem Int Ed 2006, 45:1198-215; Salmaso S, et al. J Nanosci Nanotechno 2006, 6:2736-53; Lu Y J, et al. AAPS J 2006, 8:E466-78. Overcomming the obstacles observed in this technology requires suitable protein delivery vehicles that can protect the protein cargo from denaturation and proteolysis during circulation and endocytosis; as well as shield the negatively charged protein and provide an overall positive surface charge for internalization across the phospholipid membrane (see, e.g. Yan Y, et al. ACS Nano 2010, 4:2928-36). At the same time, such protein delivery vehicles should be able to release the protein cargo in their native forms when a desired destination (e.g. the cytosol) is reached (see, e.g. Heath J R, et al. Annu Rev Med 2008, 59:251-65; Davis M E, et al. Nat Rev Drug Discov 2008, 7:771-82).

In efforts to address the challenges of intracellular protein delivery, a variety of nanoscale vehicles for cytosolic protein delivery have been designed, including lipid-based colloidal carriers (see, e.g. Zelphati O, et al. J Biol Chem 2001, 276:35103-10; Martins S, et al. Int J Nanomed 2007, 2:595-607; Mehnert W, et al. Adv Drug Deliv Rev 2001, 47:165-96; Hu F Q, et al. Int J Pharm 2004, 273:29-35), nanogels (see, e.g. Hirakura T, et al. J Control Release 2010, 142:483-9; Gu Z, et al. Nano Lett 2009, 9:4533-8; Bachelder E M, et al. J Am Chem Soc 2008, 130:10494-5; Thornton P D, et al. Adv Mater 2007, 19:1252; Kim B, et al. Biomed Microdevices 2003, 5:333-41; Murthy N, et al. Proc Natl Acad Sci U S A 2003, 100:4995-5000; Nochi T, et al. Nat Mater 2010, 9:572-8; Ayame H, et al. Bioconjug Chem 2008, 19:882-90; Lee A L, et al. Biomaterials 2008, 29:1224-32; Shu S, et al. Biomaterials 2010, 31:6039-49), micelles (see, e.g. Lee Y, et al. Angew Chem Int Ed Engl 2009, 48:5309-12; Lee Y, et al. Angew Chem Int Ed 2010, 49:1-5; Akagi T, et al. Biomaterials 2007, 28:3427-36), inorganic nanoparticles (see, e.g. Medintz I L, et al. Bioconjug Chem 2008, 19:1785-95; Ghosh P, et al. J Am Chem Soc 2010, 132:2642-5; J Am Chem Soc 2007, 129:8845-9; Bale SS, et al. ACS Nano 2010, 4:1493-500; Shimkunas R A, et al. Biomaterials 2009, 30:5720-8), nanotubes (see, e.g. Kam NWS, et al. J Am Chem Soc 2004, 126:6850-1; Crinelli R, et al. ACS Nano 2010, 4:2791-803; Kam NWS, et al. Angew Chem Int Ed 2006, 45:577-81) and protein-mediated carriers (see, e.g. Abbing A, et al. J Biol Chem 2004, 279:27410-21; Cronican J J, et al.. ACS Chem Biol 2010, 5:747-52; and Lim Y T, et al. Biomaterials 2009, 30:1197-204).

While certain conventional methods for cytosolic protein delivery have shown improved protein protection and membrane penetration, some of these techniques require covalent modification of proteins, which can disturb protein folding and impair biological activity (see, e.g. Christian D A, et al. Eur J Pharm Biopharm 2009, 71:463-74; Parveen S, et al. Clin Pharmacokinet 2006, 45:965-88). On the other hand, noncovalent carriers may exhibit low delivery efficiency and encounter difficulties due to colloidal instability (see, e.g. Ayame H, et al. Bioconjugate Chem 2008, 19(4):882-890). Furthermore, depending on the formulations, various carriers may have different intracellular fates after internalization and those with a poor endosomal escaping ability may result in localization and degradation of the therapeutic proteins in lysosomes (see, e.g. Yan Y, et al. ACS Nano 2010 May, 4(5):2928-2936). Therefore, the ability of nanocarriers to escape from endosomes is also critical for effective intracellular delivery and improved efficacy of protein therapeutics (see, e.g. Zhang Z H, et al. Angew Chem Int Ed 2009, 48:9171-5).

There is a general need for materials capable of encapsulating proteins so as to protect them in a first environment while simultaneously being capable of releasing them into a second environment. Additionally, there is a specific need for simple yet effective methods for intracellular protein delivery. The invention disclosed herein addresses these and other needs while overcoming many of the drawbacks and disadvantages of conventional methodologies.

### SUMMARY OF THE INVENTION

Embodiments of the invention include methods of making and using compositions comprising a polymer shell that encapsulates one or more polypeptides. In embodiments of the invention, the structure of the shell is designed in a manner that allows it to release the polypeptide(s) into selected environments. In typical embodiments of the invention, polymer components of the shell are interconnected by disulfide-containing crosslinked moieties, linkages which maintain the integrity of the polymer shell under certain environmental conditions such as those typically found outside of cells. Such linkages can be selected for an ability to degrade under other environmental conditions such as those that occur within the cellular cytosol. This degradation compromises the integrity of the polypeptide shell and results in the polypeptide being released from this shell. Illustrative embodiments of the invention include methods for using compositions of the invention for the intracellular delivery of polypeptides. As disclosed herein, by utilizing, for example, the redox potential differences that occur in different environments, a variety of polyeptide delivery systems can be made.

The invention disclosed herein has a number of embodiments. One embodiment of the invention is a composition of matter comprising at least one polypeptide, and a polymeric network. In this embodiment, the polymeric network is coupled together by disulfide bonds so as to form a shell that encapsulates the polypeptide. The disulfide bonds are disposed within this polymeric network in an orientation designed so that they are reduced when exposed to certain agents within an external environment, and this reduction of these bonds alters the shell in a manner that allows the polypeptide to migrate from the shell into the external environment. Typically in such embodiments, the polypeptide is entrapped within, but not coupled to the polymeric network. Optionally, the shell is spherical and has a diameter of less than 150, 125, 100, 75, 50, 25, 20, 15, 10 or 5 nanometers. In certain embodiments of the invention, the polymeric network designed to exhibit a specific material profile, for example a surface charge of between 3 and 5 millivolts at a physiological pH. In common embodiments of the invention, the polypeptide comprises a native protein, for example one that induces cellular death (e.g. apoptin). In some embodiments of the invention, the polypeptide comprises a detectable marker (e.g. a green fluorescent protein).

Another embodiment of the invention is a method of delivering a polypeptide into an intracellular environment of a cell comprising of the steps of combining the cell with a composition of matter comprising the polypeptide disposed within a polymeric network. In this embodiment, the polymeric network is crosslinked by disulfide bonds so as to form a shell that encapsulates the polypeptide. This method then comprises allowing this composition to cross a membrane of the cell and enter an intracellular environment. In the intracellular environment, the disulfides bonds of the polymeric network are then reduced in a manner that compromises the integrity of the polymer shell and allows the polypeptide to migrate from within the shell into the intracellular environment. In illustrative embodiments of the invention, the cell is a human cancer cell and the polypeptide is selected for an ability to alter a metabolic pathway of the cell. In the working embodiments disclosed in the Examples below, the polypeptide induces cellular death.

Yet another embodiment of the invention is a method of forming a modifiable polymeric nanocapsule disposed around one or more polypeptides. Typically these methods include forming a mixture comprising a polypeptide, a plurality of polymerizable monomers; and a crosslinking agent selected for its ability to form disulfide bonds that are reduced in the cytosol of a mammalian cell. In such methods the mixture is exposed to conditions that first allow the plurality of polymerizable monomers and the crosslinking agent to adsorb to surfaces of the polypeptide. Polymerization of the plurality of polymerizable monomers and the crosslinking agent at interfaces between the monomers and the polypeptide is then initiated so that the modifiable polymeric nanocapsule is formed, one that surrounds and protects the polypeptide. In working embodiments disclosed in the Examples below, the plurality of polymerizable monomers comprises an acrylamide, the crosslinking agent comprises a cystamine moiety, and polymerization is initiated by adding a free radical initiator to the mixture. In typical embodiments, the polypeptide is not covalently coupled to the polymeric nanocapsule following the polymerization of the plurality of polymerizable monomers and the crosslinking agent, and therefore free to migrate from the nanocapsule upon loss of its integrity (e.g. as a result of reduction of its disulfide bonds). Optionally, the mixture comprises a plurality of polypeptides associated within a protein complex (e.g. a multimeric apoptin complex).

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 illustrates a general depiction of the formation of redox-responsive protein nanocapsules. (A) Schematic of protein nanocapsules with redox-responsive polymeric matrix (R and R′ represent different monomers' moeities); and (B) various embodiments of chemical structures of monomers and crosslinker for disulfur (S—S) linked nanocapsule materials.

FIG. 2 illustrates various characterization of S—S CP-3 nanocapsules from example experiments: (A) Dynamic Light Scattering graphs illustrating the hydrodynamic sizes of the native CP-3 (grey), S—S CP-3 NCs (green) and S—S CP-3

NCs after degradation (blue) measured by DLS; (B) Far-UV CD spectra of native CP-3 (red), S—S CP-3 NCs (black); (C) Before degredation TEM images of S—S CP-3 NCs; and (D) After degradation TEM images of S—S CP-3 NCs after treatment with 2 mM GSH for 2 hours at 37° C.

FIG. 3 illustrates S—S CP-3 nanocapsules degradation and protein release:

(A) degradation kinetics shown by normalized scattering intensity at 2 mM GSH (red), 0.5 mM GSH (blue) and no GSH (black); and (B) activity of CP-3 released from S—S CP-3 NCs towards colorimetric substrate Ac-DEVD-pNA (8.0 mM). The absorbance of cleaved pNA was measured at 409 nm.

FIG. 4 illustrates cellular uptake and trafficking of S—S eGFP nanocapsules by HeLa cells: (A) fluorescence microscope images of eGFP nanoparticle internationalization with HeLa cells after 3 hour incubation with 400 nM S—S eGFP NCs (left) and rhodamine-CP3 internationalization with 400 nM rhodamine-tagged S—S CP-3 NCs (right). Nuclei were stained with DAPI. The scale bar is 100 μm; (B) inhibition of the cellular internalization of S—S eGFP NCs (8 nM) by HeLa cells at 4° C. The mean fluorescence intensity was measured by flow cytometry and was represented as the percentage of fluorescence at 37° C.; (C) the trafficking of S—S eGFP NCs through endosomes. Cells were incubated with 10 nM S—S eGFP NCs at 37° C. for various time periods, 0, 30, 60 and 120 min. Early endosomes were detected by early endosome antigen 1 (EEA1, red). Late endosomes were detected by cation-independent mannose-6-phosphate receptor (CI-MPR, blue). The scale bar represents 10 μm; and d) quantification of S—S eGFP NCs colocalized with EEA1+(solid) or CI-MPR+ (stripe) endosomes at various incubation times. Coloalization coefficients were calculated using Manders' overlap coefficient (>10 samples). The error bars indicate standard deviation.

FIG. 5 illustrates the viability of different cancer cell lines in the presence of S—S CP-3 NCs (i.e., cytotoxicity of S—S CP-3 nanocapsules toward different cancer cell lines). For each cell line, the cells are treated for 48 hours with native CP-3, S—S BSA NCs, nondegradable CP-3 NCs and S—S CP-3 NCs at concentrations of 50 nM, 100 nM, 200 nM, 400 nM, 800 nM and 1600 nM. Cell viability was measured by using the MTS assay. Cell lines used were: a) HeLa; b) MCF-7; and c) U-87 MG.

FIG. 6 illustrates apoptosis induced by S—S CP-3 nanocapsules: (A) bright-field-microscopy images of HeLa cells treated for 24 hours with (i) control (saline); (ii) 800 nM S—S CP-3 NCs; (iii) 800 nM nondegradable CP-3 NCs; (iv) native CP-3; and (v) 800 nM S—S BSA NCs. The scale bar represents 100 μm; and (B) apoptosis (i.e., apoptotic fragmentation of the nucleosome) detected by APO-BrdU™ TUNEL assay with treatment of 800 nM S—S CP-3 NCs for 24 hours. Red fluorescence represents the propidium-iodide-stained total DNA, and green fluorescence represents the Alexa Fluor 488-stained nick end label, the indicator of apoptotic DNA fragmentation. The merged pictures combine the PI-stained nuclei and the Alexa Fluor 488-stained nick end label. The scale bar represents 100 μm.

FIG. 7 illustrates a schematic diagram of the synthesis of degradable apoptin nanocapsules (S—S APO NC) and its delivery into tumor cells to induce apoptosis.

FIG. 8 illustrates S—S APO NC characterization and cellular localization. Shown are TEM images of (A) native MBP-APO; (B) enlarged image of MBP-APO; (C) S—S APO NC; and (D) degraded S—S APO NC after treatment with 2 mM GSH for 6 hours at 37° C.; (E) confocal microscopy of cellular localization of rhodamine-labeled MBP-APO encapsulated in redox-responsive (S—S NC) and nondegradable NC (ND NC) to cancer cell lines HeLa and MCF-7 and noncancerous HFF. Nuclei were stained with DAPI (blue). The scale bar is 20 μm.

FIG. 9 illustrates cytotoxicity and apoptosis observed following S—S APO NC delivery. Shown are graphs for (A) HeLa; (B) MCF-7; (C) MDA-MB-231; or (D) HFF cells with treatment of different concentrations of S—S APO NC, ND APO NC, and native MBP-APO. (E) Apoptosis induced by S—S APO NC as determined by TUNEL assay. Images on the left are bright field microscopy images of MDA-MB-231 and HFF cells treated for 24 hours with 200 nM S—S APO NC. The scale bar represents 50 μm. Images right of the dash line shows detection of apoptotic fragmentation of the nucleosome after same treatment using APO-BrdU™ TUNEL assay kit. The scale bar represents 50 μm. Red fluorescence represents the propidium-iodide (PI)-stained total DNA, and green fluorescence represents the Alexa Fluor 488-stained nick end label, the indicator of apoptotic DNA fragmentation. The merged pictures combine the PI-stained nuclei and the Alexa Fluor 488-stained nick end label. (Note the bright field images do not overlap with the fluorescent images; cells were detached and collected for TUNEL assay after treatment).

FIG. 10 illustrates treatment of S—S APO NC that resulted in tumor growth retardation through apoptosis. (A) Significant tumor inhibition was observed in the mice treated by S—S APO NC. Female athymic nude mice were subcutaneously grafted with MCF-7 cells and treated with intratumoral injection of MBP-APO (n=4) or S—S APO NC (n=4) (200 μg/mouse) every other day. PBS (n=3) and S—S BSA NC (n=4) were included as negative controls. The average tumor volumes were plotted vs. time. Asterisks indicate injection days. (B) Detection of apoptosis in tumor tissues after treatment with different NCs. Cross-sections of MCF-7 tumors were stained with fluorescein-dUTP (green) for apoptosis and DAPI for nucleus (blue). The scale bars represent 50 μm.

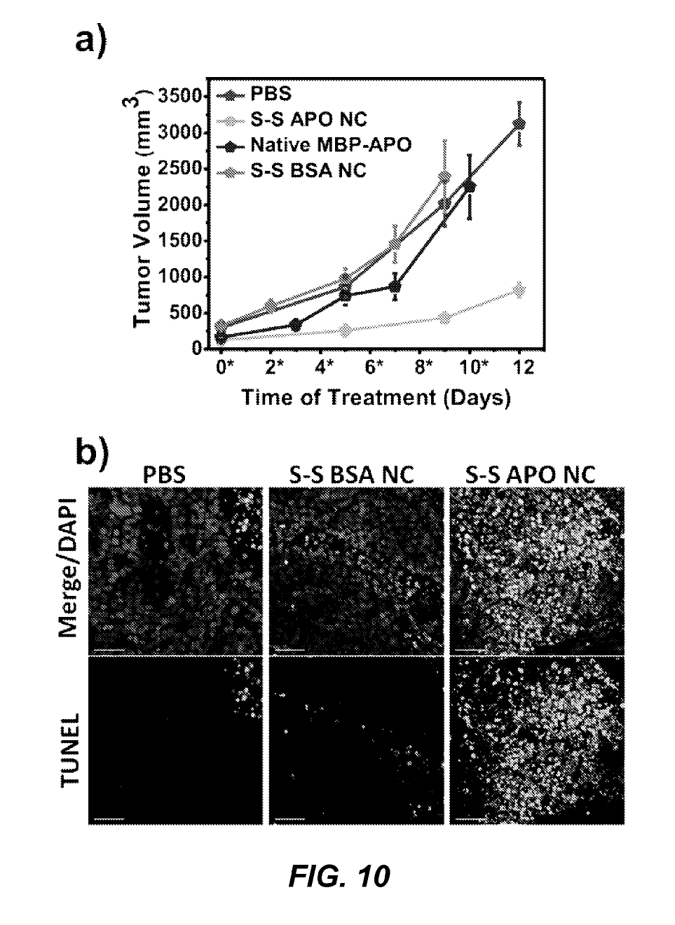

FIG. 11 illustrates a SDS-PAGE for denatured MBP-APO samples. Lane 1 depicts the molecular weight marker; Lane 2 depicts purified MBP-APO; Lane 3 depicts wash fraction; Lane 4 depicts unbounded cell lysate proteins; and Lane 5 depicts insoluble fractions.

FIG. 12 illustrates the size distribution of native MBP-APO and S—S APO NC formed. The hydrodynamic sizes of the native MBP-APO (grey) and S—S APO NC (red) were determined by DLS.

FIG. 13 illustrates internalization of S—S APO NC and ND APO NC. Fluorescent microscope images of MDA-MB-231 cells are shown after 1 and 24 hours incubation with 20 nM S—S Rho-APO NCs and with 20 nM ND Rho-APO NCs. Nuclei were stained with DAPI. The scale bars represent 50 μm.

FIG. 14 illustrates MDA-MB-231 cells TUNEL assay control groups. Left images of the dash line are Bright-field-microscopy images of MDA-MB-231 treated for 24 hours with (i) control (saline); (ii) 200 nM native MBP APO; (iii) 200 nM ND

APO NC. The scale bars represent 50 μm. Images right of the dash line are apoptotic fragmentations of the nucleosome detected by APO-BrdU™ TUNEL after the same treatment as above. The scale bars represent 50 μm. Red fluorescence represents the PI-stained total DNA, and green Alexa Fluor 488 fluorescence represents apoptotic DNA fragmentation. The merged pictures combine the PI-stained nuclei and the

Alexa Fluor 488-stained nick end label. Note the bright field images do not overlap with the fluorescent images.

FIG. 15 illustrates examples of the mean hydrodynamic size and c-potential of protein NCs.

### DETAILED DESCRIPTION OF THE INVENTION

Unless otherwise defined, all terms of art, notations and other scientific terms or terminology used herein are intended to have the meanings commonly understood by those of skill in the art to which this invention pertains. Many of the techniques and procedures described or referenced herein are well understood and commonly employed using conventional methodology by those skilled in the art. All publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further the actual publication dates may be different from those shown and require independent verification. In the description of the preferred embodiment, reference is made to the accompanying drawings which form a part hereof, and in which is shown by way of illustration a specific embodiment in which the invention may be practiced. It is to be understood that other embodiments may be utilized and structural changes may be made without departing from the scope of the present invention.

Desirable cancer therapies are both potent and specific towards tumor cells (see, e.g. J. B. Gibbs, Science 2000, 287, 1969-1973; and J. H. Atkins, L. J. Gershell, Nat Rev Drug Discov 2002, 1, 491-492). Unfortunately, many conventional small molecule chemotherapeutics do not discriminate between cancerous and normal cells, cause undesirable damage to healthy tissues, and are therefore unable to be administered at high dosage. In contrast, cytoplasmic and nuclear proteins that selectively alter the signaling pathways in tumor cells, reactivate apoptosis and restore tissue homeostasis, can eliminate cancerous cells and delay tumor progression with less collateral damage to other tissues (see, e.g. G. I. Evans, et al. Nature, 2001, 411, 342-348; J. C. Reed, Cancer Cell, 2003, 3, 17-22; T. G. Cotter, Nat. Rev. Cancer, 2009, 9, 501-507; A. Russo, et al. Ann Oncol 2006, 17, 115-123). Intracellular delivery of such proteins, including human tumor suppressors (such as p53, see, e.g. C. J. Brown, et al. Nat. Rev. Cancer, 2009, 9, 862-873) and exogenous tumor-killing (see, e.g. M. H. M. Noteborn, Euro. J. Pharmacol. 2009, 625, 165-173) proteins (such as apoptin, see, e.g. C. Backendorf, et al. Annu. Rev. Pharmacol. Toxicol. 2008, 48, 143-169; M. Los, et al. Biochem. Biophys. Acta. 2009, 1793, 1335-1342), in their functional forms is attractive as a new anti-cancer therapy modality.

The alteration of cellular processes involved in cancer is considered in a variety of therapeutic and approaches. For example, the dysregulation of apoptosis in cancer cells has been investigated extensively to reveal attractive therapeutic opportunities for cancer treatment. Mechanisms responsible for the inactiviation of the apoptosis machinery suggest that the restoration of apoptosis by delivering apoptosis-inducing proteins intracellularly can be a highly effective modality for cancer therapy. The cytosolic delivery of such proteins can potentially resurrect the apoptotic pathways and directly induce tumor cell death. However, proteins have poor membrane permeability and low serum stability, and therefore require suitable transporters for their efficient delivery. A nanoscale approach to cytosolic protein delivery is the reverse encapsulation of protein cargo in a degradable polymeric layer. The polymer shell can serve as a protective layer that shields the protein from proteases and denaturants; as well as presenting a positively charged vehicle for cellular internalization.

Direct delivery of proteins to the cytosol of cells holds tremendous potential for a variety of therapeutic and diagnostic applications. Engineering vehicles for escorting proteins to the cytosol in a controlled release fashion has thus generated considerable interest. In one aspect of the present invention, methods are disclosed for the preparation of redox-responsive single-protein nanocapsules for intracellular protein delivery. Through interfacial polymerization, the target protein is noncovalently encapsulated into a positively-charged polymeric shell interconnected by disulfide-containing crosslinkers. The dissociation of the polymeric shell under reducing conditions and the subsequent release of protein may be confirmed using cell-free assays in the presence of glutathione (GSH). In illustrative experiments shown in the Examples below, the nanocapsules were demonstrated to be efficiently internalized into the cells and to release the protein in the reducing cytosol. In this way, cellular environments and mechanisms can be harnessed to allow the selective degradation of nanocapsules and and associated release of polypeptide cargo into selected environments. Embodiments of the present invention therefore present effective intracellular protein delivery strategies for therapeutic applications (e.g. to initiate cell death as disclosed in the Examples below) as well as reprogramming applications (e.g. the differentiation of pluripotent cells as disclosed for example in U.S. Pat. No. 8,093,049).

One embodiment of the invention is a composition of matter comprising at least one polypeptide, and a polymeric network. As used herein, the term “polymeric network” or alternatively “polymeric shell” refers to one or more polymers interconnected within and/or between each other to form a mesh or shell. In typical embodiments of the invention, the polymeric network is coupled together by disulfide bonds so as to form a shell that encapsulates the polypeptide. The polymeric shell forms a nanocapsule that inhibits the ability of the polypeptide contained within it to contact agents (e.g. enzymes, substrates and the like) outside of the shell. In typical embodiments, the disulfide bonds are disposed within this polymeric network in an orientation designed so that they are reduced when exposed to certain agents within an external environment, and this reduction of these bonds alters the shell in a manner that allows the polypeptide to migrate from the shell into the external environment. As is known in the art, a disulfide bond is a covalent bond, usually derived by the coupling of two thiol groups. The linkage is also called an SS-bond or disulfide bridge. Typically, the overall connectivity is therefore P—S—S—P (where “P” is the polymer and “S” is the sulfur atom). Typically in such embodiments, the polypeptide is entrapped within, but not coupled to the polymeric network. In alternative embodiments, the polymer network is coupled to the polypeptide(s) at, collectively, at least 1, 2, 3, 4, 5, 7, 10, 15, or 20 locations.

The size of a nanocapsule may vary depending on the size and number of polypeptides in the nanocapsule and the characteristics of the polymer network. In some embodiments, the nanocapsule comprising the polypeptide and the polymeric network is from about 5 nm to about 2000 nm in length as measured along its longest axis. In some embodiments, the length of the nanocapsule is at least about 10 nm, 20 nm, 30 nm, 40 nm, 50 nm, 60 nm, 70 nm, 80 nm, 90 nm, 100 nm, 125 nm, 150 nm, 175 nm, 200 nm, 250 nm, 300 nm, 400 nm, or 500 nm. In some embodiment, the length of the nanocapsule is no more than about 50 nm, 60 nm, 70 nm, 80 nm, 90 nm, 100 nm, 125 nm, 150 nm, 175 nm, 200 nm, 250 nm, 300 nm, 400 nm, 500 nm, 600 nm, 700 nm, 800 nm, 900 nm, 1000 nm 1500 nm or 2000 nm. The nanocapsule can be of any shape, depending on the size, shape and number of the enzymes in the complex. In one embodiment, the nanocapsule is substantially round. In another embodiment, the nanocapsule is substantial oval, spherical, cylinder, or pyramid-like.

Optionally, the shell is spherical and has a diameter of less than 150, 125, 100, 75, 50, 25, 20, 15, 10 or 5 nanometers.

In certain embodiments of the invention, the polymeric network designed to exhibit a specific material profile, for example one that facilitates the crossing of cell membranes. For example, in some embodiments of the invention, polymeric network is formed from a materials selected so that the nanocapsule exhibits a positive charge at pH 6, 7 or 8. In some embodiments of the invention, polymeric network exhibits a surface charge of between 3 and 5 millivolts in an extracellular milieu in vivo or in vitro. In common embodiments of the invention, the polypeptide comprises a native protein, for example one that induces cellular death (e.g. apoptin). In some embodiments of the invention, the polypeptide comprises a transcription factor, for example one involved in the differentiation of human cells (e.g. stem cells). In some embodiments of the invention, the polypeptide comprises a detectable marker (e.g. a green fluorescent protein). In certain embodiments of the invention, the shell can encapsulate two or more different polypeptides.

Another embodiment of the invention is a method of delivering a polypeptide into an intracellular environment of a cell comprising of the steps of combining the cell with a composition of matter comprising the polypeptide disposed within a polymeric network. In this embodiment, the polymeric network is crosslinked by disulfide bonds so as to form a shell that encapsulates the polypeptide. This method comprises allowing this composition to cross a membrane of the cell and enter an intracellular environment of the cell. In this intracellular environment, the disulfides bonds of the polymeric network are then reduced in a manner that allows the polypeptide to migrate from within the shell into the intracellular environment.

In some embodiments of the invention, the cell is a human cell and the polypeptide is selected for an ability to alter a metabolic pathway of the cell and/or modulate the transcription of one or more targeted genes in the cell. In illustrative embodiments of the invention, the cell is a human cancer cell and the polypeptide is selected for an ability to alter an apoptotic pathway of the cell (see, e.g. the working examples below as well as U.S. Pat. No. 8,043,831). Illustrative non-limiting examples of metabolic pathways include purine metabolism pathway, pyrimidine metabolism pathway, alanine, aspartate and glutamate metabolism pathway, glycine, serine and threonine metabolism pathway, cysteine and methionine metabolism pathway, valine, leucine and isoleucine degradation pathway, valine, leucine and isoleucine biosynthesis pathway, lysine biosynthesis pathway, lysine degradation pathway, arginine and proline metabolism pathway, histidine metabolism pathway, tyrosine metabolism pathway, phenylalanine metabolism pathway, tryptophan metabolism pathway, phenylalanine, tyrosine and tryptophan biosynthesis pathway, beta-alanine metabolism pathway, taurine and hypotaurine metabolism pathway, phosphonate and phosphinate metabolism pathway, selenocompound metabolism pathway, cyanoamino acid metabolism pathway, D-glutamine and d-glutamate metabolism pathway, D-arginine and d-ornithine metabolism pathway, D-alanine metabolism pathway, and glutathione metabolism pathway. In the working embodiments disclosed in the Examples below, the polypeptide induces cellular death.

Embodiments of the invention include methods of forming a polymeric nanocapsule disposed around one or more polypeptides that will degrade in certain environments and release the polypetides. The working examples disclosed herein use one or more apoptosis inducing proteins encapsulated by a thin positively-charged polymer shell. Typically these methods include forming a mixture comprising a polypeptide, a plurality of polymerizable monomers; and a crosslinking agent selected for its ability to form disulfide bonds. In such methods the mixture is exposed to conditions that first allow the plurality of polymerizable monomers and the crosslinking agent to adsorb to surfaces of the polypeptide. Polymerization of the plurality of polymerizable monomers and the crosslinking agent at interfaces between the monomers and the polypeptide is then initiated so that the modifiable polymeric nanocapsule is formed, one that surrounds and protects the polypeptide. In working embodiments disclosed in the Examples below, the plurality of polymerizable monomers comprises an acrylamide. Typically, the crosslinking agent comprises a cystamine moiety (as is known in the art, disulfide bonds are commonly formed from the oxidation of sulfhydryl (—SH) groups). Optionally, polymerization is initiated by adding a free radical initiator to the mixture. In typical embodiments, the polypeptide is not covalently coupled to the polymeric nanocapsule following the polymerization of the plurality of polymerizable monomers and the crosslinking agent, and therefore free to migrate away from the nanocapsule upon loss of its integrity (e.g. as a result of reduction of its disulfide bonds). Optionally, the mixture comprises a plurality of polypeptides associated within a protein complex (e.g. a multimeric apoptin complex).

A variety of monomers can be used to form polymeric networks useful in embodiments of the invention. A monomer unit is a chemical moiety that polymerizes, forming the polymer network of the nanocapsule. In some embodiments, monomer units comprise a polymerizable group having double bond, such as a vinyl, acryl, acrylamido, alkylacryl, alkylacrylamido, methacryl or methacrylamido group. Optionally different monomers are used. The polymerizable group of the different monomer units may be the same or different, so long as they are capable of forming a co-polymer under the conditions used to form the nanocapsule. For example, vinyl and acryl groups may form co-polymers under free-radical polymerization conditions.

In general, any number of different monomer units may be used to form polymers with the polypeptides, so long as the different monomer units are all capable of forming a polymer under the conditions used to form the nanocapsule. Monomer units with different side-chains may be used to alter the surface features of the nanocapsule (e.g. surface charge). The surface features may be controlled by adjusting the ratio between different monomer units. In some embodiments, the monomers may be neutral, uncharged, hydrophilic, hydrophobic, positively charged, or negatively charged. In some embodiments, the polymer network as a whole is neutral, uncharged, hydrophilic, hydrophobic, positively charged, or negatively charged. Solubility of the nanocapsule may be adjusted, for example, by varying the ratio between charged and uncharged, or hydrophilic or hydrophobic monomer units. In some embodiments, the nanocapsule has a positive or negative charge.

In some embodiments, at least one monomer unit has a positive or negative charge at the physiological pH (˜7.4). By using monomer units having a charge at pH=7.4, the overall charge of the nanocapsule may be varied and adjusted by changing the ratio of the charged and uncharged monomer units. In some embodiments, the monomer unit has a positive charge at pH=7.4. Using positively charged monomer units enables the formation of nanocapsules having a positive charge. The charge may be adjusted by changing the ratio of neutral and positively charged monomer units.

Examples of specific monomer units and their functions include acrylamide (neutral, 1), 2-hydroxyethyl acrylate (neutral, 1), N-isopropylacrylamide (neutral, 2), sodium acrylate (negatively charged, 3), 2-acryloylamido-2-methylpropanesulfonic sodium (negatively charged, 3), allyl amine (positively charged, 4), N-(3-aminopropyl) methacrylamide hydrochloride (positively charged, 4, 5), dimethylamino ethyl methacrylate (positively charged, 5), (3-acrylamidopropyl) trimethylammonium hydrochloride (positively charged, 5), methyl acrylate (hydrophobic, 6) and styrene (hydrophobic 6). The numbers in the parentheses refer to functions: 1 to 5: hydrophilic surface and moisture retention; 2) temperature responsive; 3) negatively charged surface; 4) reactive sidechain for surface modification, 5) positive charge surface, 6) hydrophobic surface.

In embodiments of the invention, the polymer network further includes at least one type of crosslinking agent. In typical embodiments, at least one crosslinker used to form the polymeric shell is a crosslinker that forms disulfide bonds that links portions of the polymeric shell. Optionally the crosslinker is N,N′-bis(acryloyl)cystamine.

Polymerization of the modified enzymes and monomer unit(s) may use any method suitable for the polymerizable groups used on the protein and monomer unit(s) and which does not destroy the function of the protein during polymerization.

Examples of polymerization methods include photopolymerization and free-radical polymerization of double bond containing polymerizable groups. In some embodiments, the polymerization is a free radical polymerization.

Polymerization can be carried out according art accepted practices used with the selected mixture components. In some embodiments, the polymerization is carried out at room temperature, though the temperature may be increased or decreased as desired, depending on the polymerization method, so long as the function of the polypeptide is not lost during polymerization. Where degradable crosslinkers or linking groups are used, the function of the nanoparticle may be measured after degradation of the polymer coating. Reaction temperatures may be increased where the polymerization reaction occurs too slowly, or where elevated temperature is needed to initiate polymerization. Temperatures may be decreased where polymerization reactions occur too quickly.

In some embodiments, the polymerization reaction is performed in water, or aqueous buffer. Other solvents may be used as desired, so long as the solvent does not interfere with the polymerization reaction, or degrade the desired polypeptide function. Mixtures of water or aqueous buffer and organic co-solvents may also be used, if necessary to dissolve reaction components, so long as the solvent mixture does not interfere with the reaction, or damage proteins such as enzymes and the like. In some embodiments, the polymerization reaction is performed in buffer.

In some embodiments, the method of producing a nanocapsule further includes a step of modifying the surface of the nanocapsule. Sidechains of the monomer unit(s) can be present on the surface of the nanocapsule after polymerization. Monomer units having a reactive sidechain (or protected reactive sidechain) may be used to prepare the nanocapsule. The reactive sidechain does not interfere with polymerization, but may undergo further chemical modification after the nanocapsule is formed (i.e. after polymerization is completed). A protected reactive sidechain may be deprotected using standard chemical deprotection methods, then reacted with a chemical modifying agent. A reactive sidechain is treated with a chemical reagent to covalently attach the surface modifying agent to the surface of the nanocapsule. The surface modification may be a small molecule, polymer, peptide, polypeptide, protein, oligonucleotide, polysaccharide, or antibody. The surface modification may alter the solubility of the nanocapsule (e.g. by adding polyethylene glycols or other hydrophilic groups), change the surface charge of the nanocapsule (e.g. by adding charged surface modifiers), or impart an additional function to the nanocapsule, such as light-emission, cell targeting or cell penetration. Examples of small molecule surface modifications include light emitting compounds, such as fluorescein, or rhodamine, or cell targeting compounds such as folic acid. Polymers include polyethylene glycol to increase solubility. Peptides and polypeptides may be used for cell targeting, and may include antibodies selective to specific cell surface features, cell signaling peptides, or hormones. Other peptides may be used to increase cell penetration of the nanocapsule (such as TAT or antennepedia homeodomain). In some embodiments, the surface modification is an antibody. Because nanocapsule can have an easily derivatizeded surface, specific antibodies can be conjugated with nanocapsules providing extra ability of targeting delivery.

The nanocapsules comprising a polymer network and a polyeptide as discussed herein can be formulated into various compositions, for use in diagnostic or therapeutic treatment methods. The compositions (e.g. pharmaceutical compositions) can be assembled as a kit. Generally, a pharmaceutical composition of the invention comprises an effective amount (e.g., a pharmaceutically effective amount) of a composition of the invention.

A composition of the invention can be formulated as a pharmaceutical composition, which comprises a composition of the invention and pharmaceutically acceptable carrier. By a “pharmaceutically acceptable carrier” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. For a discussion of pharmaceutically acceptable carriers and other components of pharmaceutical compositions, see, e.g., Remington's Pharmaceutical Sciences, 18 th ed., Mack Publishing Company, 1990. Some suitable pharmaceutical carriers will be evident to a skilled worker and include, e.g., water (including sterile and/or deionized water), suitable buffers (such as PBS), physiological saline, cell culture medium (such as DMEM), artificial cerebral spinal fluid, or the like. A pharmaceutical composition or kit of the invention can contain other pharmaceuticals, in addition to the compositions of the invention. The other agent(s) can be administered at any suitable time during the treatment of the patient, either concurrently or sequentially.

One skilled in the art will appreciate that the particular formulation will depend, in part, upon the particular agent that is employed, and the chosen route of administration. Accordingly, there is a wide variety of suitable formulations of compositions of the present invention. One skilled in the art will appreciate that a suitable or appropriate formulation can be selected, adapted or developed based upon the particular application at hand. Dosages for compositions of the invention can be in unit dosage form. The term “unit dosage form” as used herein refers to physically discrete units suitable as unitary dosages for animal (e.g. human) subjects, each unit containing a predetermined quantity of an agent of the invention, alone or in combination with other therapeutic agents, calculated in an amount sufficient to produce the desired effect in association with a pharmaceutically acceptable diluent, carrier, or vehicle. One skilled in the art can easily determine the appropriate dose, schedule, and method of administration for the exact formulation of the composition being used, in order to achieve the desired effective amount or effective concentration of the agent in the individual patient.

The dose of a composition of the invention, administered to an animal, particularly a human, in the context of the present invention should be sufficient to effect at least a detectable amount of a diagnostic or therapeutic response in the individual over a reasonable time frame. The dose used to achieve a desired effect will be determined by a variety of factors, including the potency of the particular agent being administered, the pharmacodynamics associated with the agent in the host, the severity of the disease state of infected individuals, other medications being administered to the subject, etc. The size of the dose also will be determined by the existence of any adverse side effects that may accompany the particular agent, or composition thereof, employed. It is generally desirable, whenever possible, to keep adverse side effects to a minimum. The dose of the biologically active material will vary; suitable amounts for each particular agent will be evident to a skilled worker.

Another embodiment of the invention is a kit useful for any of the methods disclosed herein, either in vitro or in vivo. Such a kit can comprise one or more of the compositions of the invention. Optionally, the kits comprise instructions for performing the method. Optional elements of a kit of the invention include suitable buffers, pharmaceutically acceptable carriers, or the like, containers, or packaging materials. The reagents of the kit may be in containers in which the reagents are stable, e.g., in lyophilized form or stabilized liquids. The reagents may also be in single use form, e.g., in single dosage form.

As noted above, specific illustrative embodiments of the invention include methods for protein delivery using nanocapsules consisting of a redox-cleavable crosslinker. Such nanocapsules are designed to open and release a polypeptide cargo into selected environments such as those where disulfide bonds are reduced to sulfhydril groups. For example, the cytosol has a low redox potential due to the abundance of reduced glutathione (GSH) in the millimolar concentration range, whereas the extracellular glutathione concentration falls in the micromolar range (see, e.g. Meister A, et al. Annu Rev Biochem 1976, 45:559-604). Glutathione reduces disulfide bonds by serving as an electron donor. In the process, glutathione is converted to its oxidized form glutathione disulfide (GSSG), also called L(-)-Glutathione.

In order to utilize redox potential differential, a variety of gene delivery systems based on dissociation of disulfide bonds have been reported (see, e.g. Bauhuber S, et al. Adv Mater 2009 Sep 4, 21(32-33):3286-3306; Takae S, et al. J Am Chem Soc 2008 May 7, 130(18):6001-6009; You Y Z, et al. J Control Release 2007 Oct. 8, 122(3):217-225; Kakizawa Y, et al. Biomacromolecules 2001 Sum, 2(2):491-497; and Lee Y, et al. Bioconjug Chem 2007 Jan.-Feb., 18(1):13-18). Embodiments of the present invention utilize disulfide-forming crosslinkers that interconnect over the protein to form a anaocapsule (NC) through interfacial polymerization. Typically the disulfuide bonds in the linkers maintain the integrity of the thin polymer shell under oxidative conditions outside the cell, but undergo a rapid degradation and cargo release after entry into reductive conditions such as those that occur in the cytosol. As demonstrated in Examples below, using such strategies, native proteins can be efficiently delivered into various mammalian (e.g. human) cell lines.

In various embodiments of the invention, redox-responsive nanocapsule compositions can be used to shuttle different protein targets useful for biomedical applications, including cancer therapy, vaccination, regenerative medicine, treating loss-of-function genetic diseases and imaging (e.g. imaging useful in diagnostic methodologies). For example, proteins that can lead to programmed cell death in cancer cells, such as the tumor suppressor p53, or tumor-selective killing proteins, will be similarly formulated in the methods described herein and be delivered to tumors as anticancer therapeutics.

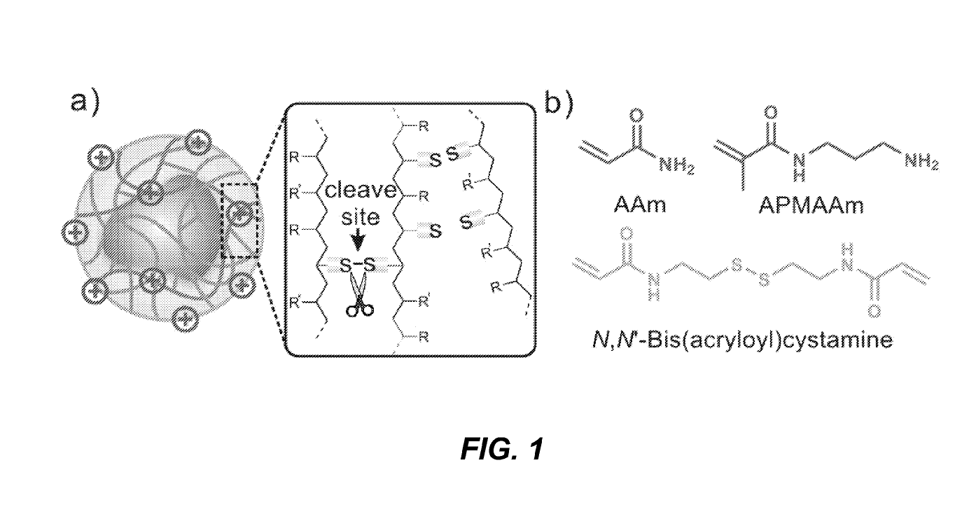

One illustrative synthesis method for single-protein NCs is schematically shown in FIG. 1A. Briefly, the target protein, which is either enhanced green fluorescent protein (eGFP), bovine serum albumin (BSA) or mature caspase 3 (CP-3), is mixed with acrylamide (AAm), positively-charged N-(3-Aminopropyl) methacrylamide (APMAAm) and the crosslinker. After the monomers are allowed to electrostatically adsorb onto the surface of the protein, in situ polymerization is initiated by the addition of free radical initiators. To render the crosslinking of the capsule reversible under reducing conditions, a S—S crosslinker, such as a cleavable disulfide-bond containing N,N′-bis(acryloyl)cystamine is used (FIG. 1b). When needed as a control as for the CP-3 studies, the target protein can be encapsulated using the nondegradable crosslinker N,N′-methylene bisacrylamide. Using this interfacial polymerization strategy, no covalent bond is formed between the resulting polymeric shell matrix and the core target protein, which ensures that the native protein is released upon degradation. Following polymerization and encapsulation, the NCs may be further purified from unreacted monomers using filters, such as AMICON centrifugal filters (molecular weight cutoff 30 kDa) and buffer exchanged into PBS buffer.

Embodiments of the S—S crosslinked NCs disclosed herein are simultaneously designed to be rapidly degraded when treated with physiologically relevant concentrations of GSH, to be internalized into cells and to escape from endosomes, so as to deliver functional proteins. In one exemplary implementation of this embodiment of the invention, CP-3 delivered using S—S NCs was able to induce apoptosis in human cancer cell lines including HeLa, MCF-7 and U-87 MG. Illustrative experiments using CP-3 as a model protein have demonstrated that CP-3 encapsulated in the S—S NCs can be internalized into a wide variety of cancer cell lines; can be delivered into cytosol upon entry; and can be released in functional forms so as to trigger apoptosis in the target cells via native CP-3 functions. Such results with the CP-3 NCs demonstrate the potential of using such nanocarriers to delivery protein-based cancer therapeutics. As shown by the experimental results presented herein, the redox-responsive encapsulation strategy is confirmed to be a simple yet effective method of intracellular protein delivery. Embodiments of the invention provide new and significant strategies for intracellular delivery of functional proteins through the redox gradient of cellular environment.

Embodiments of the invention can incorporate any one of a wide variety of polypeptides known in the art, for example, one selected for an ability to alter a metabolic pathway of the cell and/or modulate the transcription of one or more targeted genes in the cell. In the working examples below, a polypeptide encapsulated by a polymer shell of the invention is apoptin. Apoptin has been investigated widely as an anti-tumor therapeutic option because of its high potency in inducing tumor-selective apoptosis (see, e.g. C. Backendorf, et al. Annu. Rev. Pharmacol. Toxicol. 2008, 48, 143-169). Different gene therapy approaches have been used to administer apoptin to mouse xenograft tumor models, in which significant reduction in tumor sizes and prolonged lifespan of mice have been observed without compromising the overall health (see, e.g. A. M. Pietersen, et al. Gene Ther 1999, 6, 882-892; M. M. van der Eb, et al. Cancer Gene Ther. 2002, 9, 53-61; D. J. Peng, et al. Cancer Gene. Ther. 2007, 14, 66-73). However, as with other gain-of-function therapy candidates, in vivo gene delivery approaches, such as the use of viral vectors may lead to genetic modifications and elicit safety concerns (see, e.g. M. L. Edelstein, et al. Gene. Med. 2007, 9, 833-842). While protein transduction domain (PTD)-fused apoptin has been delivered to cells (see, e.g. L. Guelen, et al. Oncogene, 2004, 23, 1153-1165; J. Sun, et al. S. Du, Int. J. Cancer, 2009, 124, 2973-2981), this approach suffers from inefficient release of the cargo from endosomes and instability of the unprotected protein. Development of nanoparticle carriers to aid the delivery of functional forms of apoptin to tumor cells is therefore desirable (see, e.g. J. Shi, et al. Nano Lett. 2010, 10, 3223-3230). In this context, another embodiment of the invention is a method for the delivery of apoptin to cancer cells using a degradable polymeric nanocapsule.

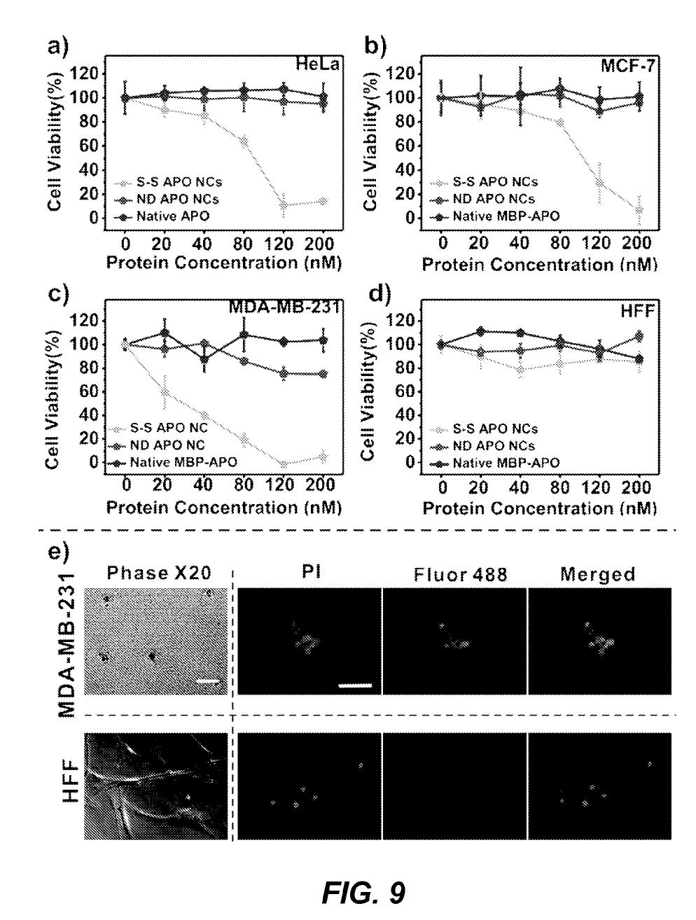

In another illustrative embodiment of the invention shown in the Examples, apoptin, a tumor-selective killer, is encapsulated into a polymeric nanocapsule with a redox-responsive crosslinker to be delivered intracellularly. The data presented herein (see, e.g. that shown in FIG. 9) illustrates the efficient delivery of apoptin using a degradable polymeric nanocapsule to different cancer cell lines in vitro and to xenograft tumor models in vivo. Apoptin is a 121-residue protein derived from chicken anemia virus (see, e.g. C. Backendorf, et al. Annu. Rev. Pharmacol. Toxicol. 2008, 48, 143-169 and U.S. Pat. No. 7,566,548). When transgenically expressed, apoptin was shown to induce p53-independent apoptosis in a variety of tumor and transformed cells (see, e.g. S. M. Zhuang, et al. Cancer Res 1995, 55, 486-489; J. G.

Teodoro, et al. Genes Dev. 2004, 18, 1952-1957), while leaving normal and untransformed cells unaffected (see, e.g. A. A. A. M. Danen-Van Oorschot, et al. Proc Natl Acad Sci USA 1997, 94, 5843-5847). Apoptin exists in a globular multimer complex of thirty to forty subunits with no well-defined secondary structure (see, e.g. S. R. Leliveld, et al. J Biol. Chem. 2003, 278, 9042-9051). While the exact mechanism of the tumor selectivity is unresolved, apoptin is known to translocate to the nuclei where tumor-specific phosphorylation at residue Thr108 takes place, leading to accumulation of apoptin in nuclei and activation of the apoptotic cascade in tumor cells (see, e.g. A. A. A. M. Danen-Van Oorschot, et al. J. Biol. Chem., 2003, 278, 27729-27736). In normal cells, apoptin is not phosphorylated at Thr108 and is located mostly in the cytoplasm, where it becomes aggregated and degraded (see, e.g. J. L. Rohn, et al. J. Biol. Chem. 2002, 277, 50820-50827). As shown in the Examples, using embodiments of the invention, recombinant apoptin can be released in its native form in cytoplasm to induce tumor-specific apoptosis and inhibit tumor growth, as demonstrated in both in vitro and in vivo studies.

In one exemplary implementation, a maltose-binding-protein fused apoptin (MBP-APO) is used. Such fusion proteins can be used with embodiments of the invention to allow, for example, high expression of fusion proteins in soluble form from Escherichia coli, whereas native apoptin form inclusion bodies (see, e.g. S. R. Leliveld, et al. J Biol. Chem. 2003, 278, 9042-9051). MBP-APO, although five times the length as native apoptin in primary sequence, has been shown by gel filtration to similarly assemble into a multimeric complex and to capture the essential functions and selectivity of native apoptin (see, e.g. S. R. Leliveld, et al. J Biol. Chem. 2003, 278, 9042-9051). Nanoparticle-mediated delivery of functional MBP-APO poses unique challenges (see, e.g. Z. Gu, A. Biswas, M. Zhao, Y. Tang, Chem. Soc. Rev. 2011, 40, 3638-3655). First, the protein cargo preassembles into large complexes with an average diameter of ˜40 nm and molecular weight of ˜2.4 MDa (see, e.g. S. R. Leliveld, et al. J Biol. Chem. 2003, 278, 9042-9051). To achieve nanoscale sizes that are optimal for in vivo administration (˜100 nm) (see, e.g. P. P. Adiseshaiah, et al. Nanomed. Nanobi. 2010, 2, 99-112), a loading strategy that leads to compact particles is preferred. Second, in order to maintain the noncovalent multimeric state of functional MBP-APO, the protein loading and releasing events need to take place under very mild, physiological conditions in the absence of surfactants. The globular and undefined nature of MBP-APO further complicates the preparation process. Lastly, the protein cargo must be released into the cytoplasm in its native functional form, including the correct spatial presentation of nuclear localization/export signals, as well as the phosphorylation site and downstream signaling elements within apoptin.

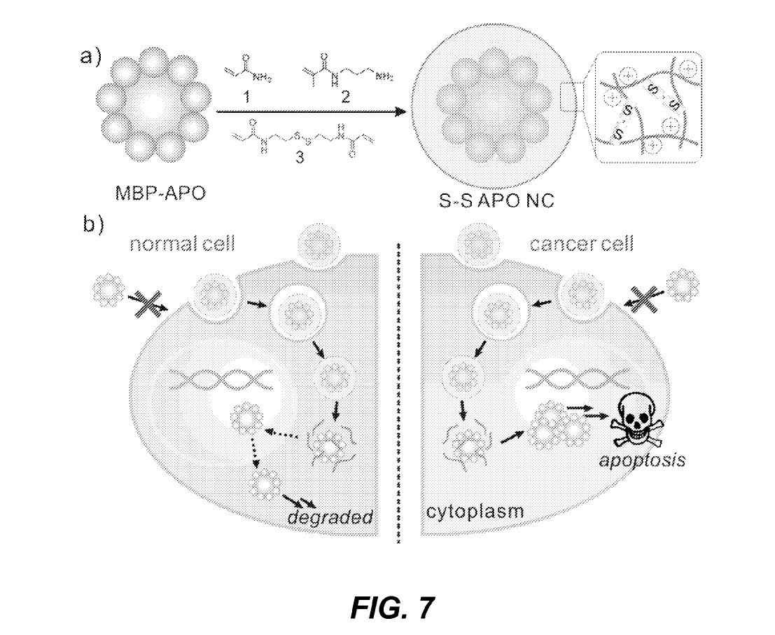

Based on these challenges and requirements, a polymeric nanocapsule strategy is used (see, e.g. M. Zhao, et al. Biomaterials 2011, 32, 5223-5230) for the functional delivery of MBP-APO, in which the protein complex is nearly individually and noncovalently protected in a water soluble polymer shell (FIG. 7). This slightly positively-charged shell protects the MBP-APO from serum proteases and harsh environment, as well as enables its cellular uptake through endocytosis (see, e.g. Z. Gu, et al. Nano Lett. 2009, 9, 4533-4538). The polymeric layer is weaved together by redox-responsive crosslinkers containing disulfide bond (S—S), which is degraded once the nanocapsules are exposed to the reducing environment in cytoplasm. This characteristic of the polymer shell ensures completely reversible encapsulation and release of native protein in the cell.

### EXAMPLES

Now, the present invention will be described in detail in reference to various illustrative examples, but is be limited to these examples.

### Example 1

### Illustrative Nanocapsules Comprising a Caspase 3 Polypeptide (CP-3 NC)

In addition to the disclosure below, illustrative methods and materials useful in embodiments of the invention are disclosed in Zhao M, et al. Redox-responsive nanocapsules for intracellular protein delivery. Biomaterials 32 (2011):5223-5230, the contents of which are incorporated by reference.

### Illustrative Materials and Methods

Illustrative Materials Useful with Embodiments of the Invention

N-(3-aminopropyl) methacrylamide hydrochloride was purchased from

Polymer Science, Inc. CellTiter 96® AQueous One Solution Cell Proliferation Assay (MTS) reagent was purchased from Promega Corporation. APO-BrdU™ TUNEL Assay Kit was purchased from Invitrogen. All other chemicals were purchased from Sigma-Aldrich and used as received. The deionized water was prepared by a Millipore NanoPure purification system (resistivity higher than 18.2 MΩ cm⁻¹).

Illustrative Instruments Useful with Embodiments of the Invention

The Bradford protein assay was carried out on a Thermo Scientific GENESYS 20 spectrometer. Caspase 3 (CP-3) proteolysis activity was measured using a Beckman Coulter DU® 520 spectrometer. Far-UV circular dichroism (CD) spectra of proteins were tested using JASCO J-715 Circular Dichroism spectrometer. The size distributions and zeta potentials of NCs were measured on the Malvern particle sizer Nano-ZS. Transmission electron microscopy (TEM) images were obtained using Philips EM-120 TEM instrument. Fluorescent images were taken with Zeiss Axio Observer Z1 Inverted Microscope and Yokogawa spinning-disk confocal microscope (Solamere Technology Group, Salt Lake City, Utah) on Nikon eclipse Ti-E Microscope equipped with a 60×1.49 Apo TIRF oil objective and a Cascade II: 512 EMCCD camera (Photometrics). An AOTF (acousto-optical tunable filter) controlled laser-merge system (Solamere Technology Group Inc.) was used to provide illumination power at each of the following laser lines: 491 nm, 561 nm, and 640 nm solid-state lasers (50 mW for each laser). FACScan and FACSort (BD Bioscience) were used for flow cytometry analysis.

### Illustrative Methods Useful with Embodiments of the Invention

### Protein Expression and Purification

The plasmid pHC332 for expression of the mature CP-3 was a generous gift from Dr. A. Clay Clark (North Carolina State University). Plasmid pHC332 was transformed into Escherichia coli BL21(DE3) cells and incubated at 37° C. overnight on LB agar plate with 100 μg/mL ampicillin. Colonies were picked and grown overnight at 37° C. with shaking (250 rpm) in 5 mL ampicillin-containing LB media. Overnight cultures were then inoculated in 1 L of LB media with 100 μg/mL ampicillin and allowed to grow under 37° C. until the absorbance of cell density (OD₆₀₀) reached 1.0. Isopropyl β-D-1-thiogalactopyranoside (IPTG) was added to a final concentration of 0.1 mM to induce protein expression. After overnight incubation at 16° C., the E. coli cells were harvested by centrifugation (2000 g, 4° C., 15 min). Cell pellets were then resuspended in 30 mL Buffer A (50 mM Tris-HCl, pH 8.0, 2 mM dithiothreitol, 2 mM EDTA) and lysed by sonication. Cell debris and insoluble proteins were removed by centrifugation (20,000 g, 4° C., 30 min), followed by the addition of 1 mL Ni-NTA resin (Qiagen) into the cleared cell lysate and a binding period of 3 h at 4° C. Afterward, the protein was then purified on a gravity column using Buffer A with increasing concentrations of imidazole (10, 20, and 250 mM). The protein concentration was qualitatively assessed by SDS-PAGE and quantitatively determined by the Bradford protein assay using bovine serum albumin (BSA) as the standard. Enhanced green fluorescence protein (eGFP) was prepared following the procedures described above, except for the induction was carried out when OD₆₀₀ reached 0.4.

### Single-protein Encapsulation

The concentration of protein (CP-3, eGFP and BSA) was diluted to 1 mg/mL with 5 mM sodium bicarbonate buffer at pH 9. Then 200 mg/mL acrylamide (AAm) monomer was added to 1 mL of protein solution with stirring at 4° C. After 10 min, the second monomer, N-(3-aminopropyl) methacrylamide (APMAAm), was added while stirring. Different crosslinkers, N,N′-methylene bisacrylamide for nondegradable (ND) NCs and N,N′-bis(acryloyl)cystamine for disulfide-crosslinked NCs, was added 5 min after the addition of APMAAm. The polymerization reaction was immediately initiated by adding 30 mL of ammonium persulfate (100 mg/mL, dissolved in deoxygenated and deionized water) and 3 μL of N,N,N′,N′-tetramethylethylenediamine. The polymerization reaction was allowed to proceed for 60 min. The molar ratio of AAm/APMAAm/crosslinker was adjusted to 12:9:1. Buffer exchange with phosphate-buffered saline (PBS) buffer (pH 7.4) was used to remove the remaining monomers and initiators. Rhodamine-tagged CP-3 NCs was obtained through encapsulation of CP-3 modified with 5-Carboxy-X-rhodamine N-succinimidyl ester (mass ratio (CP-3: rhodamine): 4:1).

Characterization of protein NCs

Samples of NCs (0.05 mg/mL) for TEM imaging were negatively stained with 2% uranyl acetate in alcoholic solution (50% ethanol). The lamella of stained sample was prepared on carbon-coated electron microscopy grids (Ted Pella, Inc.). The degradation process of S—S NCs was dynamically monitored by dynamic light scattering (DLS) in PBS buffer. Different amounts of GSH were combined with 1 mg/mL S—S NCs in PBS buffer, to obtain final GSH concentrations of 0.5 mM and 2 mM. The average count rates at different time points were continuously monitored for 90 min at 25° C. The release of CP-3 from S—S NCs and its activity were assessed using colorimetric substrate peptide Ac-DEVD-pNA (p-nitroanilide). Samples with 0.01 mg native CP-3 or S—S CP-3 NCs incubated with different concentrations of GSH (0.2 mM, 0.5 mM, 1 mM and 2 mM) were prepared in 1.0 mL PBS buffer. With the addition of 32 μM Ac-DEVD-pNA, the intensity of cleaved pNA was spectrometrically recorded at 409 nm for 120 min.

### Cellular Uptake, Internalization Pathway and Trafficking

HeLa cells (ATCC, Manassas, Va.) were cultured in Dulbecco's Modified Eagle's Media (DMEM) (Invitrogen) supplemented with 10% bovine growth serum (Hyclone, Logan, Utah), 1.5 g/L sodium bicarbonate, 100 μg/mL streptomycin and 100 U/mL penicillin, at 37° C. with 98% humidity and 5% CO₂. To visualize NCs uptake, cells were seeded into 48-well plate, with a density of 5000 cells/well in 250 μL of media with supplements. Once the confluency of ˜50-60% was reached, S—S NCs with eGFP and rhodamine-tagged CP-3 were added to a final concentration of 400 nM. After 3 h of incubation, cells were washed with PBS twice, stained with DAPI Nucleic Acid Stain (Invitrogen) and imaged. To determine the internalization pathway of S—S NCs, HeLa cells were seeded into 12-well plates at a density of 50,000 cells/well. The plates were incubated at 37° C. overnight. The media was then replaced with 0.5 mL of fresh media containing 8 nM S—S eGFP NCs. After incubating at 4° C. and 37° C. for 2 h, each well was washed with PBS and the cells were trypsinized and collected in PBS. After fixation using 2% paraformaldehyde, samples were analyzed via FACS with a 488 nm argon laser. The signal from the FL1 bandpass emission (530/30) was used for eGFP. Markers for different endosome stages were used for internalization trafficking A concentration of 10 nM S—S eGFP NCs was added to HeLa cells at 4° C. for 30 min. The plates were moved to 37° C. and incubated for 30 min, 1 and 2 h. Cells were then fixed with 4% paraformaldehyde, permeabilized with 0.1% Triton X-100, and stained with antibodies, mouse anti-EEA1 antibody against early endosomes and rabbit anti-CI-MPR antibody against late endosomes (Cell Signaling Technology, Inc.). Texas red goat anti-mouse IgG and Alexa Fluor® 647 goat anti-rabbit IgG (Invitrogen) were added as the secondary antibody.

### Cytotoxicity Assays

Different cancer cells, HeLa, MCF-7 and U-87 MG cells (ATCC, Manassas, Va.), were seeded into 96-well plates, each well containing 5000 cells in 100 μL of DMEM with supplements. Different concentrations of protein and NCs were added into each well and the plates were incubated at 37° C. with 98% humidity and 5% CO₂ for 48 h. The cells were washed with PBS solution twice and 100 μL of fresh cell culture media with supplements was added. Then 20 μL MTS solution (CellTiter 96® AQueous One Solution Cell Proliferation Assay, Invitrogen) was added into each well and the plates were incubated for 3 h at 37° C. The absorbance of product was read at 490 nm using a microplate reader (PowerWave X, Bio-tek Instruments, USA).

### TUNEL Assays

Apoptosis of HeLa cells was detected using APO-BrdU Terminal Deoxynucleotidyl Transferase dUTP Nick End Labeling (TUNEL) assay kit. Cells were seeded at a density of 100,000 cells/well into a 6-well plate in 2 mL of cell culture media with supplements. Proteins or NCs were added after the confluency of ˜50-60% was reached. After 24 h of incubation, cells were fixed with 1% paraformaldehyde in PBS, followed by the addition of DNA labeling solution containing terminal deoxynucleotidyl transferase and bromodeoxyuridine (BrdUrd). Cells were then stained with Alexa Fluor® 488 dye-labeled anti-BrdUrd antibody.

Samples were deposited onto slides, which were later stained with propidium iodide (PI) solution containing RNase A. Images were obtained by fluorescence microscope (Zeiss, Observer Z1) using appropriate filters for Alexa Fluor 488 and PI.

### Formation and Characterization of Protein NCS

The synthesis method for single-protein NCs is schematically shown in FIG. 1a. Briefly, the target protein, which is either enhanced green fluorescent protein (eGFP), bovine serum albumin (BSA) or mature caspase 3 (CP-3), is mixed with acrylamide (AAm), positively-charged N-(3-Aminopropyl) methacrylamide (APMAAm) and the crosslinker. After the monomers are allowed to electrostatically adsorb onto the surface of the protein, in situ polymerization is initiated by the addition of free radical initiators. To render the crosslinking of the capsule reversible under reducing conditions, we choose to use the cleavable disulfide-bond containing N,N′-bis(acryloyl)cystamine (referred to as S—S crosslinker) (FIG. 1b). When needed as a control as for the CP-3 studies, the target protein is also encapsulated using the nondegradable crosslinker N,N′-methylene bisacrylamide. Using this interfacial polymerization strategy, no covalent bond is formed between the resulting polymeric shell matrix and the core target protein, which ensures that the native protein is released upon degradation. Following polymerization and encapsulation, the NCs were purified from unreacted monomers using AMICON centrifugal filters (molecular weight cutoff 30 kDa) and buffer exchanged into PBS buffer.

The surface charges of the NCs weaved with S—S crosslinkers (referred to as S—S NC) were assessed to be between 3.6 and 4.7 mV, confirming the necessary positive surface charge desired for cellular internalization (Table 1).

TABLE 1
Mean hydrodynamic size and zeta potential of protein NCs

| | Abbreviation | | | |
|---|---|---|---|---|
| | S-S CP-3 | S-S BSA | S-S eGFP | ND CP-3 |
| | NCs | NCs | NCs | NCs |
| Size (nm) | 11.3 | 10.0 | 9.9 | 9.5 |
| Zeta potential (mV) | 3.6 ± 0.1 | 4.7 ± 0.4 | 3.6 ± 0.7 | 4.0 ± 0.4 |

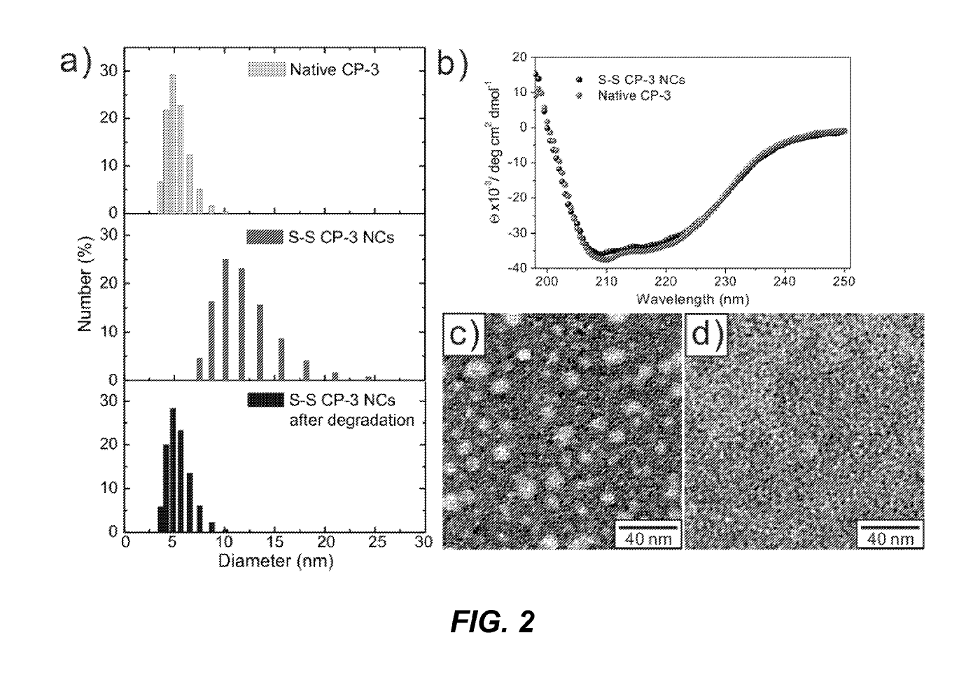

The hydrodynamic sizes of the various NCs were measured by Dynamic Light Scattering (DLS) and are shown in FIG. 2a and Table 1. Whereas native CP-3 protein had an average diameter of 5 nm, S—S NCs containing CP-3 had an average diameter of 11.3 nm with a relatively narrow size distribution. Similar sizes S—S NCs encapsulating eGFP and BSA were also observed. The narrow size distribution of S—S NCs was further confirmed by TEM, in which the NCs adopted a robust and consistent spherical shape in aqueous solution (FIG. 2c). To ensure that the encapsulation process does not affect the folding of CP-3, circular dichroism was used to compare the secondary structures of native and encapsulated CP-3. As shown in FIG. 2b, the two spectra both show the characteristic minima (208 and 222 nm) expected for the predominantly a-helical CP-3. The nearly overlapping spectra validate that the secondary structure of CP-3 was well preserved during the encapsulation process.

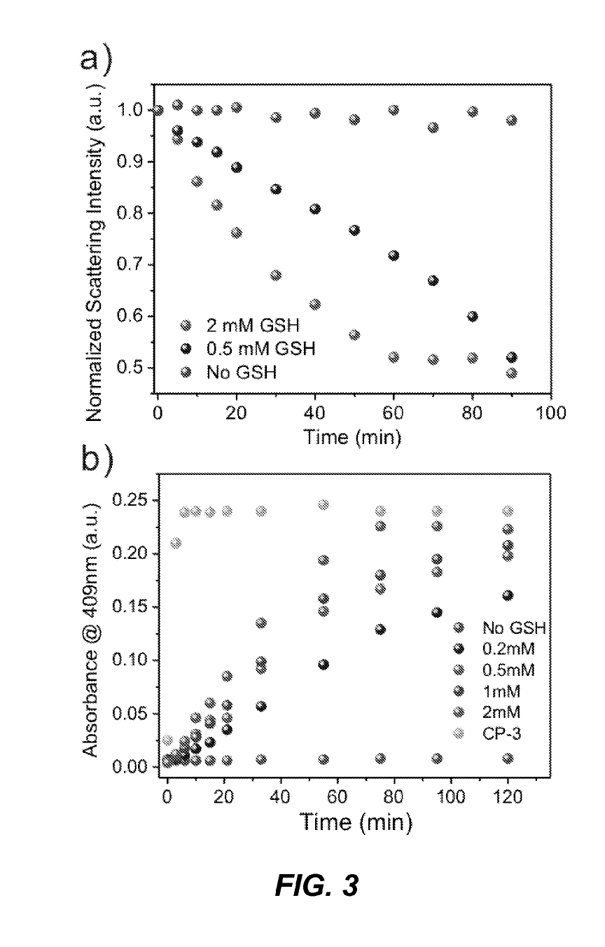

To determine if the S—S NCs are degradable under reducing conditions, we treated S—S CP-3 NCs with the physiologically relevant GSH at 37° C. As expected, after treatment with 2 mM GSH for 2 h, the hydrodynamic size of the NCs decreased to an average size of 5.3 nm, which was highly consistent with that of native CP-3 protein before encapsulation (FIG. 2a). This result suggests that the NCs have been nearly completely degraded upon reduction of the disulfide bonds. The degradation of S—S NCs was further substantiated by TEM, as no spherical particles were visible after GSH treatment (FIG. 2d). To evaluate the kinetics of the disassembly process, we monitored the time-dependent decrease in the hydrodynamic sizes in the presence of different amount of GSH (FIG. 3a). The relative scattering intensity of S—S CP-3 NCs decreased steadily in the presence of GSH, suggesting the continuous decrease of the particle size or the number of particles. At a GSH concentration as low as 0.5 mM, which is the lower limit of estimated intracellular concentration of GSH (see, e.g. Meister A, et al. Glutathione and related gamma-glutamyl compounds: biosynthesis and utilization. Annu Rev Biochem 1976, 45:559-604), CP-3 S—S NCs appeared to be completely degraded within 90 min, judged from the size decrease. At 2 mM GSH concentration, a much faster degradation process was observed. These results therefore strongly suggest that once delivered to the cytosol, the S—S NCs are expected to rapidly release the cargo protein under reducing conditions.

To test the activity of encapsulated and released CP-3, we used a colorimetric assay employing a CP-3 peptidyl substrate mimic, Ac-DEVD-pNA (p-nitroanilide) (see, e.g. Talanian RV, et al. Substrate specificities of caspase family proteases. J Biol Chem 1997, 272:9677-82). As shown in FIG. 3b, in contrast to free CP-3 protein that rapidly cleaved the substrate, CP-3 encapsulated in S—S NCs did not display any protease activity over the assay period in the absence of GSH. This result confirms that the in situ polymerization process completely shields CP-3 from the outside environment and that the peptidyl substrate is therefore inaccessible to the enzyme. This result also demonstrates that during storage and assay of the CP-3 S—S NCs, no significant diffusion of CP-3 across the polymer matrix, or spontaneous reduction of the S—S crosslinker occurs. When the degradation of the polymer shell was triggered by addition of GSH, CP-3 activity was readily observed and the rate of proteolysis was linearly correlated with the amount of reducing agent present. In the presence of 2 mM GSH, complete digestion of the substrate can be observed within 1 h. The controlled release of encapsulated CP-3 in the presence of GSH demonstrates the redox-responsiveness of the S—S NCs and also confirms the activity of the encapsulated protein is minimally affected during the entire assembly/disassembly process.

### Cellular Uptake, Internalization Pathway and Trafficking of S—S NCS

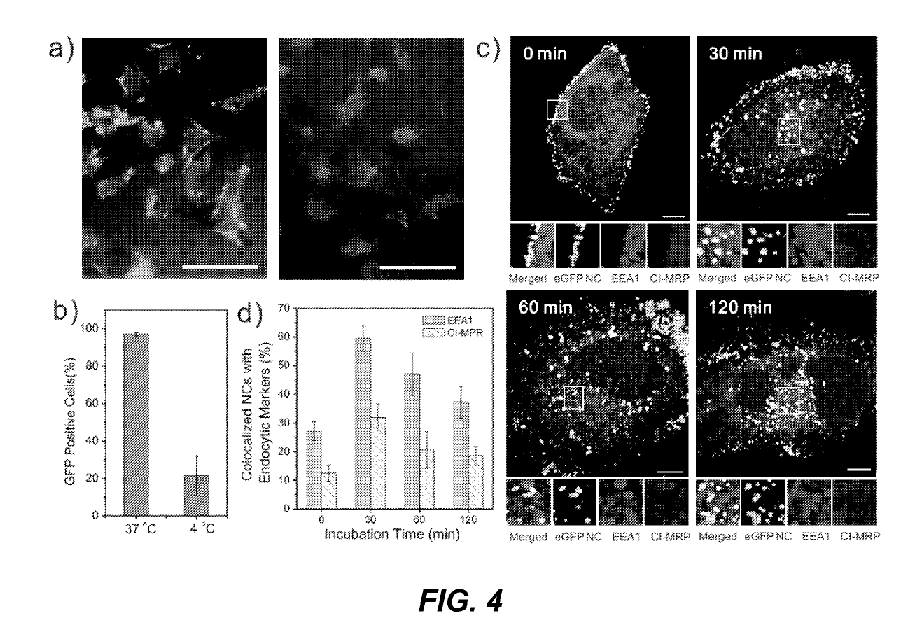

Having confirmed the desired physical properties and chemical responsiveness of the S—S NCs, we next investigated the cellular uptake and trafficking mechanisms of NCs using eGFP as a fluorescent marker. The physical characteristics of the S—S eGFP NCs are shown in Table 1. Fluorescent microscopy image of HeLa cells incubated with 400 μM eGFP S—S NCs for 3 h in media is shown in the left panel of FIG. 4a. Compared to native eGFP, which cannot penetrate the cellular membrane, eGFP encapsulated in S—S NCs appears to be efficiently internalized by the cells and the eGFP fluorescent signals were diffusively visible in the cytosol. To investigate the mechanism of NC cellular internalization, HeLa cells were incubated with S—S eGFP NCs at different temperatures and analyzed by flow cytometry. The mean intensity of eGFP fluorescence at 4° C. dropped to ˜20% of that at 37° C. (FIG. 4b), indicating the likely involvement of the energy-dependent endocytosis for NC cellular uptake. The cellular trafficking of the internalized S—S eGFP NCs was then investigated for 2 h by tracking the eGFP fluorescence at different time points and monitoring colocalization using markers for early and late endosomes (FIG. 4c). At the onset of the internalization process, all the eGFP signals were localized at the membrane of the cells as expected (0 min). Overlap of eGFP (green) with early endosomal marker EEA1 (red) at ˜60% colocalization was observed after 30 min of incubation, confirming that S—S eGFP NCs were trafficked into early endosomes upon cellular entry. While the degree of colocalization of eGFP and EEA1 signals decreased after 60 and 120min, no significant colocalization of eGFP with late endosomal marker CI-MPR (blue) was detected, strongly suggesting that some of the NCs or proteins have been delivered into the cytosol (FIG. 4c and d). Results from these imaging studies validate that S—S NCs can indeed be internalized by cells and at least a significant portion of the internalized NCs and the cargo can escape from the endosomal compartment and reach the desired destination. Combined with the rapid degradation rate of the S—S NCs in the presence of GSH, release of the protein cargo into the cytosol of target cells can be expected to be highly efficient and completed within hours after the onset of internalization.

### Apoptosis is Observed Following Delivery of CP-3 S—S NCS

After confirming that NCs can be trafficked into the cytosol of cells, we next investigated the delivery of CP-3 as a functional protein using the redox-responsive NCs. CP-3 is a serine protease that can trigger rapid apoptosis, which is the desired phenotype upon successful delivery. Therefore, the S—S CP-3 NCs must be degraded once internalized to allow CP-3 to interact with its cytosolic macromolecular targets. The successful delivery of proteins—such as CP-3 to tumor cells—can also be a powerful method to resurrect a dysfunctional apoptotic pathway and directly induce tumor cell death (see, e.g. Ford KG, et al. Protein transduction: an alternative to genetic intervention? Gene Ther 2001, 8:1-4; Bale SS, et al. Nanoparticle-mediated cytoplasmic delivery of proteins to target cellular machinery. ACS Nano 2010, 4:1493-500). To verify if S—S CP-3 NCs can indeed be internalized into cells, CP-3 was first tagged with NHS-modified rhodamine dye and was then encapsulated into an S—S NC. Delivery of the tagged CP-3 NCs into HeLa cells resulted in the appearance of dispersed red color throughout the cytosol after 3 h of incubation (FIG. 4a, right).

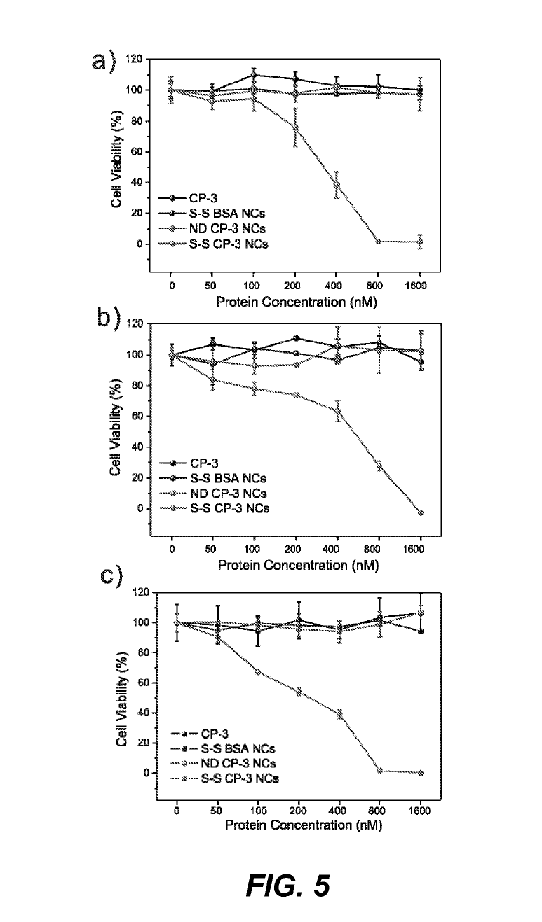

To evaluate the apoptotic potency of the designed NCs, HeLa cells were treated with S—S CP-3 NCs together with negative control samples: 1) native CP-3, which cannot be internalized; 2) S—S BSA NCs, which cannot trigger the apoptosis pathway; and 3) CP-3 encapsulated in nondegradable NCs, which shield CP-3 and prevent it from interacting with its substrates. After 48 h of treatment, the cytotoxicity of the different protein and NCs samples was assessed using the MTS assay. As shown in FIG. 5a, HeLa cells treated with S—S CP-3 NCs exhibited prominent cell death and had an IC50˜300 nM. In comparison, cells treated with each of the three control samples did not display significant cell death. The robust cell viability of the S—S BSA NCs also illustrates the polymeric material that constitutes the delivery vehicle does not have significant cytotoxicity toward human cell lines. To demonstrate the S—S NCs can be delivered to a variety of cell lines, breast cancer cell line MCF-7 (FIG. 5b) and brain cancer cell line U-87 MG (FIG. 5c) were also treated with the different NCs. Similar to the assay results from HeLa cells, both cell lines treated with S—S CP-3 NCs for 48 h showed prominent cell death, but remained viable when treated with the three control NCs. The S—S CP-3 NCs displayed an IC50 value of ˜300 nM and ˜600 nM toward U-87 MG and MCF-7 cells, respectively.

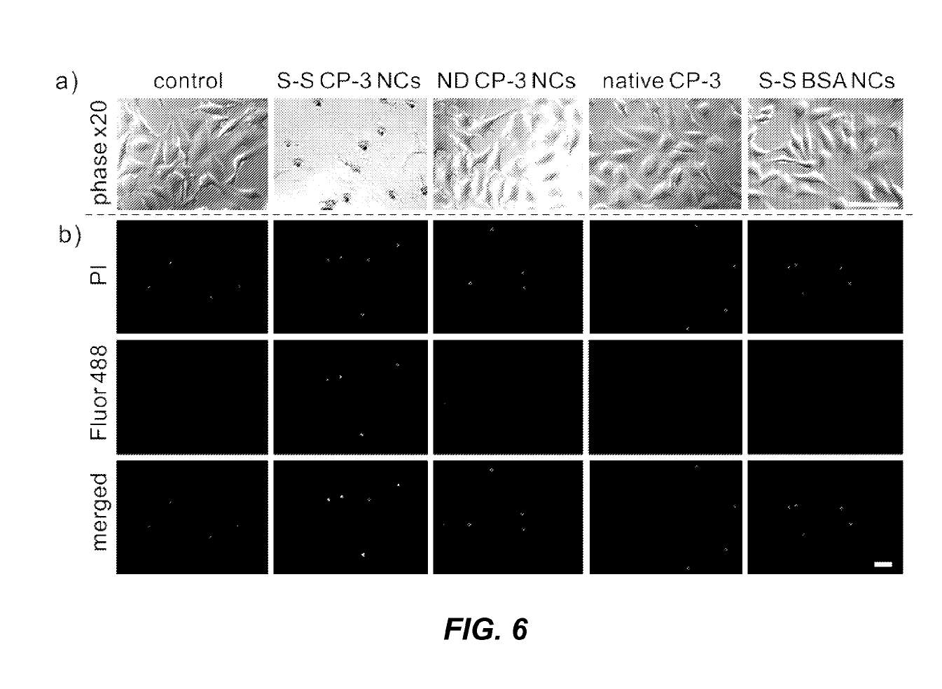

To confirm the cell death incurred after treatment with S—S CP-3 NCs was indeed apoptosis, we examined the cell morphology under bright field microscopy. As shown in FIG. 6a, only HeLa cells incubated with S—S CP-3 NCs showed apoptotic properties such as membrane blebbing and cell shrinkage. In contrast, no morphology change was observed in cells treated with control proteins or NCs. Another signature feature of apoptosis is the fragmentation of the nucleosome upon CP-3 cleavage of the caspase-activated deoxyribonuclease inhibitor (ICAD) (see, e.g. Cotter T G. Apoptosis and cancer: the genesis of a research field. Nat Rev Cancer 2009, 9:501-7), which can be detected by the TUNEL assay (see, e.g. Gavrieli Y, et al. Identification of programmed cell death in situ via specific labeling of nuclear DNA fragmentation. J Cell Biol 1992, 119:493-501). To visualize nicked DNA, the cells were detached, fixed and stained with Alexa Fluor 488 (green), while total cellular DNA was stained with propidium iodide (red). As shown in FIG. 6b, HeLa cells treated with 800 nM S—S CP-3 NCs underwent extensive apoptotic DNA fragmentation. In contrast, cells treated with the negative control samples did not display any apoptosis characteristics. Collectively, these results demonstrate that CP-3 encapsulated in the S—S NCs can 1) be internalized into the various cancer cell lines; 2) be delivered into cytosol upon entry; and 3) be released in functional forms and trigger apoptosis. Our results with the CP-3 NCs further demonstrate the potential of using nanocarriers to delivery protein-based cancer therapeutics (see, e.g. Peer D, et al. Nanocarriers as an emerging platform for cancer therapy. Nat Nanotechnol 2007, 2:751-60). Other proteins that can lead to programmed cell death in cancer cells, such as the tumor suppressor p53 (see, e.g. Joerger A C, et al. Structure-function-rescue: the diverse nature of common p53 cancer mutants. Oncogene 2007, 26:2226-42) and tumor-selective killing proteins (see, e.g. Noteborn M H. Proteins selectively killing tumor cells. Eur J Pharmacol 2009, 625:165-73), may be similarly formulated in the methods described in this work and be delivered to tumors as potential anticancer therapeutics.

### Example 2

### Illustrative Apoptin Nanocapsules (APO NC) Working Embodiments of the Invention

Illustrative Materials Useful with Embodiments of the Invention

N-(3-aminopropyl) methacrylamide hydrochloride was purchased from Polymer Science, Inc. CellTiter 96® AQueous One Solution Cell Proliferation Assay (MTS) reagent was purchased from Promega Corporation. APO-BrdU™ TUNEL Assay Kit was purchased from Invitrogen. In Situ Cell Death Detection Kit, POD; was purchased from Roche Applied Science. Female athymic nude (nu/nu) mice, 6 weeks of age, were purchased from Charles River Laboratories (Wilmington, Mass.). All other chemicals were purchased from Sigma-Aldrich and used as received. The deionized water was prepared by a Millipore NanoPure purification system (resistivity higher than 18.2 MΩ.cm⁻¹).

Illustrative Instruments Useful with Embodiments of the Invention

The Bradford protein assay was carried out on a Thermo Scientific GENESYS 20 spectrometer. The size distribution and ζ-potential of NCs were measured on the Malvern particle sizer Nano-ZS. Transmission electron microscopy (TEM) images were obtained using Philips EM-120 TEM instrument. Fluorescent images were taken with Zeiss Axio Observer Z1 Inverted Microscope and Yokogawa spinning-disk confocal microscope (Solamere Technology Group, Salt Lake City, Utah) on Nikon eclipse Ti-E Microscope equipped with a 60×1.49 Apo TIRF oil objective and a Cascade II: 512 EMCCD camera (Photometrics). An AOTF (acousto-optical tunable filter) controlled laser-merge system (Solamere Technology Group Inc.) was used to provide illumination power at each of the following laser lines: 491 nm, 561 nm, and 640 nm solid state lasers (50 mW for each laser).

Illustrative Methods Useful with Embodiments of the Invention

### Protein Expression and Purification

The pMa1TBVp3 plasmid for expression of the MBP-APO was a generous gift from Dr. C. Backendorf and Dr. M. Noteborn (Leiden University). MBP-APO plasmid was transformed into Escherichia coli BL21(DE3) cells and incubated at 37° C. overnight on LB agar plate with 100 μg/mL ampicillin. Colonies were picked and grown overnight at 37° C. with shaking (250 rpm) in 5 mL ampicillin-containing LB media. Overnight cultures were then inoculated in 500 mL of TB media with 100 μg/mL ampicillin and allowed to grow under 37° C. until the absorbance of cell density (OD₆₀₀) reached 1.0. Isopropyl β-D-1-thiogalactopyranoside (IPTG) was added to a final concentration of 0.1 mM to induce protein expression. After overnight incubation at 16° C., cells were harvested by centrifugation (2,000 g, 4° C., 15 min). MBP-APO protein was purified according to procedure described in previous literature (see, e.g. S. R. Leliveld, et al., J Biol. Chem. 2003, 278, 9042-9051). Cell pellets were first resuspended in 30 mL lysis buffer (25 mM Tris-HCl, 500 mM NaC1, 10% glycerol pH 7.4) and lysed by sonication. Cell debris and insoluble proteins were removed by centrifugation (17,000 rpm, 4° C., 30 min), followed by filtering through 0.22 μm filters to clear the cell lysate further. Protein was then purified on an amylase column (New England BioLabs), which was passed over 5 times with lysate under gravity flow at 4° C. then washed with wash buffer (20 mM Tris-HCL, 50mM NaC1, 1 mM EDTA, pH 7.4) to remove unbounded protein. MBP-APO was eluted from the column with 10 mM maltose buffer and buffer exchanged into PBS. The protein concentration was qualitatively assessed by SDS-PAGE and quantitatively determined by the Bradford protein assay using bovine serum albumin (BSA) as the standard.

### NC Preparation

The concentration of protein was diluted to 1 mg/mL with 5 mM sodium bicarbonate buffer at pH 9. Then 200 mg/mL acrylamide (AAm) monomer was added to 1 mL of protein solution with stirring at 4° C. After 10 min, the second monomer, N-(3-aminopropyl) methacrylamide (APMAAm), was added while stirring. Different crosslinkers, N,N′-methylene bisacrylamide for ND NC and N,N′-bis(acryloyl)cystamine for S—S NC, was added 5 min after the addition of APMAAm. The polymerization reaction was immediately initiated by adding 30 μL of ammonium persulfate (100 mg/mL, dissolved in deoxygenated and deionized water) and 3 μL of N,N,N′,N′-tetramethylethylenediamine. The polymerization reaction was allowed to proceed for 60 min. The molar ratio of AAm/APMAAm/cross-linker was adjusted to 12:9:1. Buffer exchange with phosphate-buffered saline (PBS) buffer (pH 7.4) was used to remove the remaining monomers and initiators. Rhodamine-labeled APO NCs was obtained through encapsulation of MBP-APO modified with 5-Carboxy-X-rhodamine N-succinimidyl ester (mass ratio (MBP-APO: rhodamine): 4:1).

### Characterization of APO NCs

The mean hydrodynamic size and zeta potential of NC were determined by DLS in PBS buffer. Samples of NCs (0.05 mg/mL) for TEM imaging were negatively stained with 2% uranyl acetate in alcoholic solution (50% ethanol). The lamella of stained sample was prepared on carbon-coated electron microscopy grids (Ted Pella, Inc.).

### Cellular Uptake and Localization

MDA-MB-231, HeLa, MCF-7, and HFF cells (ATCC, Manassas, Va.) were cultured in Dulbecco's Modified Eagle's Media (DMEM) (Invitrogen) supplemented with 10% bovine growth serum (Hyclone, Logan, Utah), 1.5 g/L sodium bicarbonate, 100 μg/mL streptomycin and 100 U/mL penicillin, at 37° C. with 98% humidity and 5% CO₂. To visualize NCs uptake, MDA-MB-231 cells were seeded into 48-well plate, with a density of 10,000 cells/well in 250 μL of media with supplements. S—S Rho-APO NC and ND Rho-APO NC were added to a final concentration of 20 nM. After 1 hour and 24 hours of incubation, cells were washed with PBS twice, stained with DAPI Nucleic Acid Stain and imaged. To determine the cellular localization of protein delivered, confocal images were taken with HeLa, MCF-7, and HFF cells incubated with 20nM of S—S Rho-APO NC or ND Rho-APO NC at 37° C. for 24 hours. Nuclei were then counterstained with DAPI. The Z stack images of cells were imaged at 0.4-μm intervals and analyzed by Nikon NIS Element software. Fluorescent images were acquired on a Yokogawa spinning-disk confocal scanner system (Solamere Technology Group, Salt Lake City, Utah) using a Nikon eclipse Ti-E microscope equipped with a 60×/1.49 Apo TIRF oil objective and a Cascade II: 512 EMCCD camera (Photometrics, Tucson, Ariz., USA). An AOTF (acousto-optical tunable filter) controlled laser-merge system (Solamere Technology Group Inc.) was used to provide illumination power at each of the following laser lines: 491 nm, 561 nm, and 640 nm solid state lasers (50 mW for each laser).

### Cytotoxicity Assay

Different cancer cells lines, HeLa, MCF-7 and MDA-MB-231, as well as noncancerous HFF, were seeded into 96-well plates, each well containing 5,000 cells in 100 μL of DMEM with supplements. Different concentrations of protein and NCs were added into each well and the plates were incubated at 37° C. with 98% humidity and 5% CO₂ for 48 hours. The wells were washed with PBS solution twice and 100 μL of fresh cell culture media with supplements was added. Then 20 μL MTS solution (CellTiter 96® AQueous One Solution Cell Proliferation Assay, Invitrogen) was added into each well and the plates were incubated for 3 hours at 37° C. The absorbance of product was read at 490 nm using a microplate reader (PowerWave X, Bio-tek Instruments, USA).

In vitro TUNEL Assay

Apoptosis of cells was detected using APO-BrdU Terminal Deoxynucleotidyl Transferase dUTP Nick End Labeling (TUNEL) assay kit. MDA-MB-231 and HFF cells were seeded at a density of 100,000 cells/well into a 6-well plate in 2 mL of cell culture media with supplements. Proteins and NCs were added after cells covered 80% of bottom surface. After 24 hours of incubation, cells were fixed with 1% paraformaldehyde in PBS, followed by the addition of DNA labeling solution containing terminal deoxynucleotidyl transferase and bromodeoxyuridine (BrdUrd). Cells were then stained with Alexa Fluor® 488 dye-labeled anti-BrdUrd antibody. Samples were deposited onto slides, which were later stained with propidium iodide (PI) solution containing RNase A. Images were obtained by fluorescence microscope (Zeiss, Observer Z1) using appropriate filters for Alexa Fluor 488 and PI.

In Vivo Studies with MCF-7 Xenograft Model

All mice were housed in an animal facility at the University of Southern California in accordance with institute regulations. Female athymic nude (nu/nu) mice were subcutaneously grafted on the back flank with 5×10⁶ MCF-7 tumor cells. Afterwards, tumor size was monitored by a fine caliper and the tumor volume was calculated as the product of the two largest perpendicular diameters and the vertical thickness (L×W×D, mm³). When the tumor volume reached 100-200 mm³, mice were randomly separated into different groups. From day 0, mice were treated with intratumoral injection of native MBP-APO or S—S APO NC (200m per mouse) every other day. PBS and S—S BSA NC were included as the negative controls. When the tumor volume oversized 2500 mm³, the mice were euthanized by CO₂ according animal protocol. The average of tumor volume was plotted as the tumor growth curve in respective treated groups.

### Histology Study

For histology study, treated tumor samples were collected and fixed in 4% paraformaldehyde, and processed for staining using the In Situ Cell Death Detection Kit. The stained tumor slides were observed under microscope, and representative pictures were taken for analysis. Paraformaldehyde-postfixed frozen tumor sections (5-μm thick) were permeabilized with 0.1% triton X-100 and stained with a TUNEL assay kit (In Situ Cell Death Detection Kit, POD; Roche Applied Science, Indianapolis, Id.) in accordance with the manufacturer's instructions. DAPI was used for nuclear counterstaining.

### Results and Discussion

Based on these challenges and requirements, we selected a polymeric nanocapsule strategy (see, e.g. M. Zhao, et al. Biomaterials 2011, 32, 5223-5230) for the functional delivery of MBP-APO, in which the protein complex is nearly individually and noncovalently protected in a water soluble polymer shell (FIG. 7).

This slightly positively-charged shell protects the MBP-APO from serum proteases and harsh environment, as well as enables cellular uptake through endocytosis (see, e.g. Z. Gu, et al. Nano Lett. 2009, 9, 4533-4538). The polymeric layer is weaved together by redox-responsive crosslinkers containing disulfide bond(S—S), which can be degraded once the nanocapsules are exposed to the reducing environment in cytoplasm. The noncovalent aspects of the polymer shell ensures completely reversible encapsulation and release of native protein in the cell.

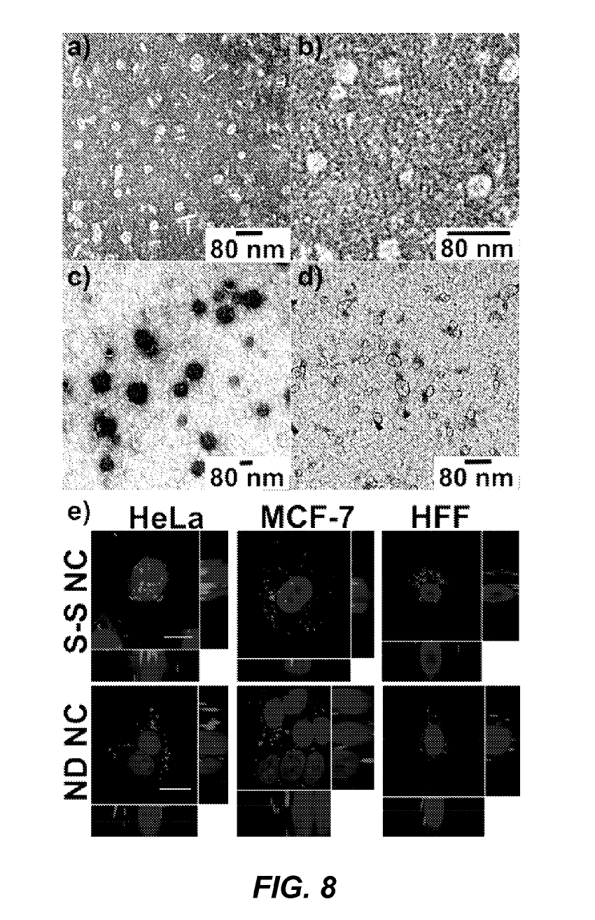

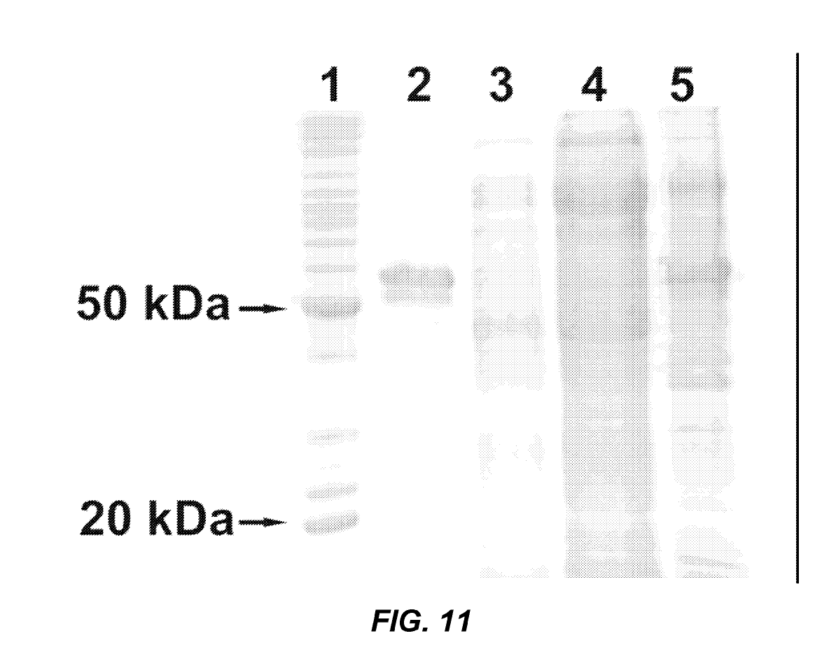

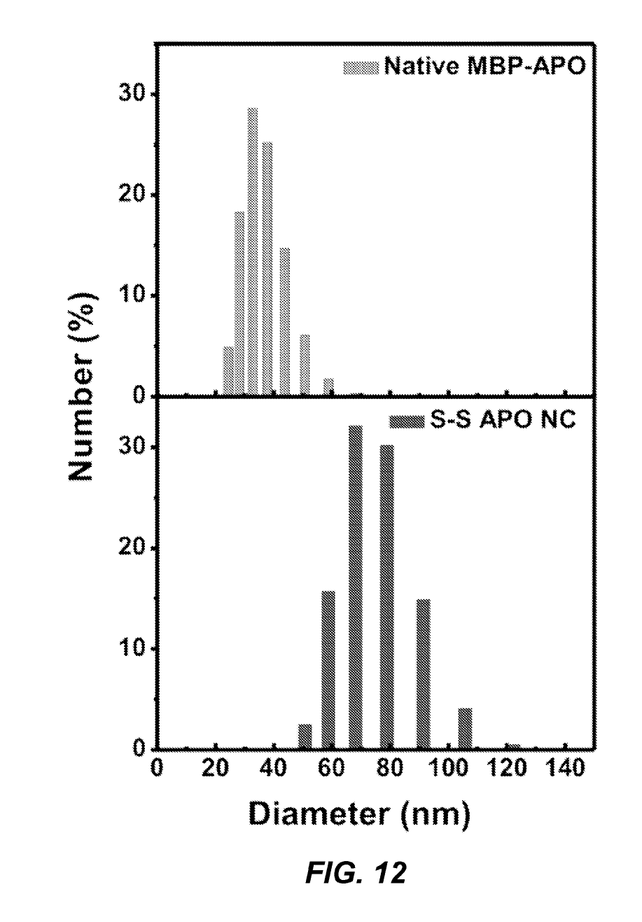

MBP-APO (pI=6.5) was first purified from E. coli extract using an amylose-affinity column (Example 2, FIG. 11). Dynamic Light Scattering (DLS) measurement revealed an average hydrodynamic radius of 36.1 nm (distribution shown in FIG. 12), consistent with the reported size for the MBP-APO complex (see, e.g. S. R. Leliveld, et al. J Biol. Chem. 2003, 278, 9042-9051). Transmission Electron Microscopy (TEM) analysis of MBP-APO showed similarly sized protein complexes (FIG. 8a and enlarged in FIG. 8b). Interestingly, MBP-APO complexes appear to adopt a disk-shaped structure despite the lack of defined secondary structure from the apoptin component. Since the apoptin portion of the protein can self-assemble into the ˜40-mer complex, we propose a three dimensional arrangement of MBP-APO in which the C-terminal apoptin forms the central spoke of the wheel-like structure (FIG. 7), with the larger MBP portion distributed around on the edge. The planar arrangement allows the apoptin portion of the fusion protein to remain accessible to its protein partners, which may explain how the MBP-APO fusion retains essentially all of the observed functions of native apoptin.

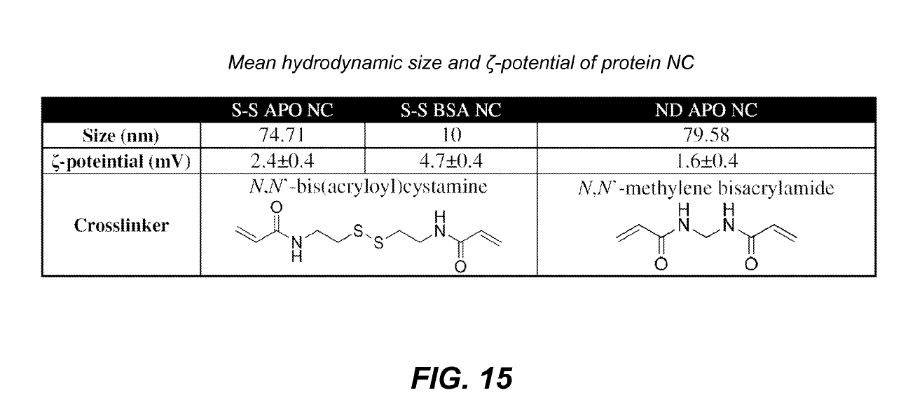

Following electrostatic deposition of the monomers acrylamide (1 in FIGS. 7a) and N-(3-aminopropyl)methacrylamide (2), and the crosslinker N,N′-bis(acryloyl)cystamine (3), at a molar ratio of 12:9:1, onto MBP-APO (1 mg) in carbonate buffer (5 mM, pH 9.0), in situ polymerization was initiated with the addition of free radical initiators and proceeded for one hour. The molar ratio reported is optimized to minimize protein aggregation and precipitation, as well as to maximize the solution stability of the nanocapsule formed (S—S APO NC). Excess monomers and crosslinkers were removed using ultrafiltration and the S—S APO NC was stored in PBS buffer (pH 7.4). DLS clearly showed increase in average diameter of the sample to ˜75 nm with a slightly positive c-potential value of 2.4 mV (FIG. 15). TEM analysis of the S—S APO NC confirmed the nearly doubling in diameter of the spherical particle (FIG. 8c). Unexpectedly, the NCs displayed dark contrast upon uranyl acetate staining, which hints that the cores of the particles are very densely packed. As expected from the redox-responsive crosslinkers, the reduction of nanocapsule size to essentially that of the free MBPAPO can be seen upon treatment of the reducing agent glutathione (GSH) (2 mM, 6 hours, 37° C.). As shown in FIG. 8d, the densely packed NCs were completely dissociated into particles (˜30 nm) that resemble those seen in FIG. 8a, confirming the reversible nature of the encapsulation process. In contrast, the densely packed MBP-APO nanocapsules crosslinked with the nondegradable crosslinker N,N′-methylene bisacrylamide (ND APO NC) were not degraded in the presence of GSH (data not shown).

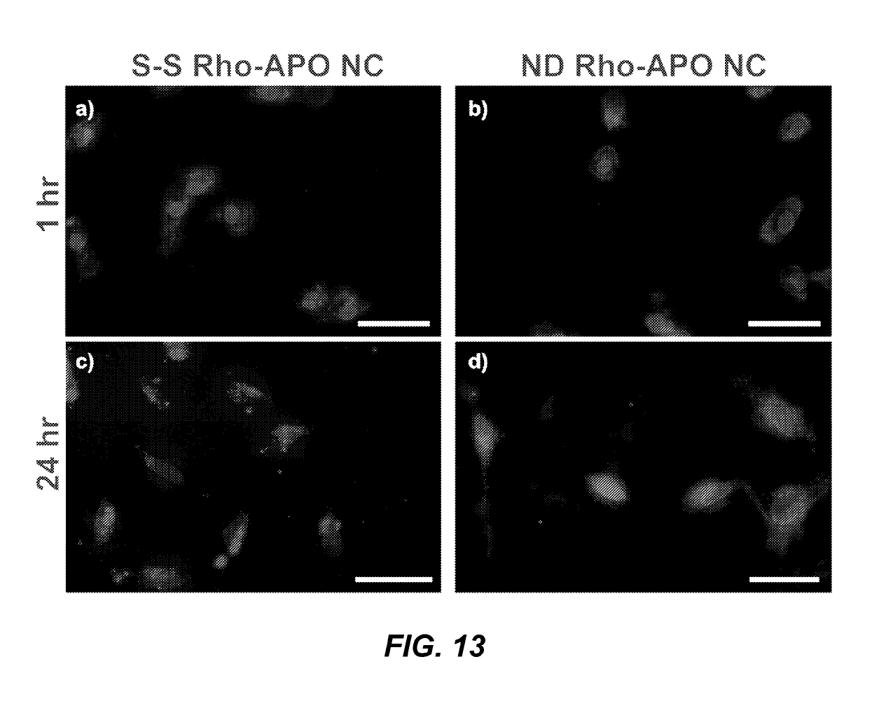

After demonstrating the in situ polymerization strategy can reversibly wrap the large MBP-APO complex, we next examined the cellular uptake of the S—S APO NC and cellular localization of the cargo. If the unique tumor selectivity of MBP-APO is maintained following the encapsulation and release processes, we expect to find MBP-APO to enter the nucleus of the tumor cell, whereas in noncancerous cell it resides in the cytoplasm. Prior to the polymerization process, the MBP-APO protein was conjugated to amine-reactive rhodamine (Rho-APO) (Supporting information). Subsequent encapsulation yielded similarly sized NCs. Two cancer cell lines HeLa and MCF-7, together with the human foreskin fibroblast (HFF), were treated with either S—S Rho-APO NC or ND Rho-APO NC. Fluorescent images showed all NCs readily penetrated the cell membrane and can be observed to be localized in the cytoplasm within one hour (FIG. 13). To analyze protein localization using confocal microscopy, cells were fixed and the nuclei were stained with DAPI (FIG. 8e). As cytotoxicity was observed with the cancer cell lines treated with S—S APO NC (see FIG. 9), sub-lethal concentrations (20 nM) of the NCs were added to image samples and the cells were collected before morphology changed and detachment took place. In the case of ND Rho-APO NCs, red fluorescence signals remained in the cytoplasm for all three cell lines, indicating the MBP-APO was well-shielded by the nondegradable polymer shell and its nuclear localization signals is not accessible to the transport machinery. In stark contrast, when HeLa cells were treated with S—S

Rho-APO NC, strong red fluorescence of rhodamine was localized in the nuclei, resulting in intense pink color as a result of overlapping of rhodamine and DAPI fluorescence. Z-stacking analysis confirmed the MBP-APO to be localized inside of the nuclei (FIG. 8e). Similar results were observed with MCF-7 cells, although the fluorescence intensity was not as strong as in the HeLa cells. These results confirmed that the MBP-APO delivered can indeed be released in native forms inside the cytoplasm and enter the nuclei. More importantly, the specificity of MBO-APO delivered towards cancer cell lines were demonstrated in the confocal image of noncancerous HFF cells treated with S—S Rho-APO NC, as all of the dye signals remained in the cytoplasm and no nuclear accumulation can be observed.

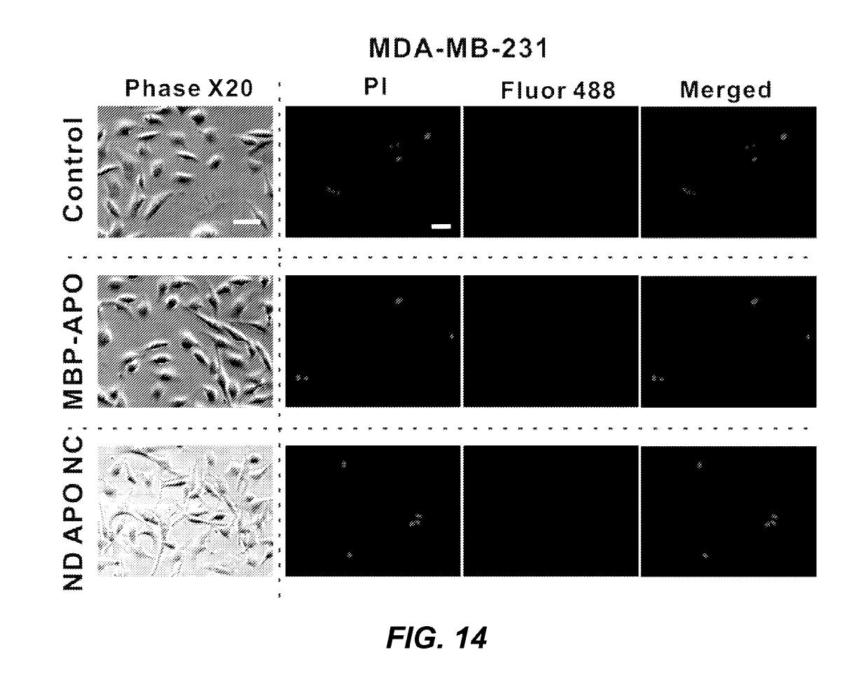

We then investigated whether the MBP-APO protein delivered still possess its function to induce tumor-selective apoptosis. The potency and selectivity of S—S APO NC were tested on various cell lines including HeLa, MCF-7, MDA-MB-231, and HFF (FIG. 9ad). MTS assay was used to measure cell viability 48 hours after addition of the protein and NC. For each cell line, ND APO NC and native MBP-APO were used as negative controls. When S—S APO NC was added to a final concentration of 200 nM, all three cancer cell lines had no viable cells, whereas ˜75% of the HFF had survived. The IC50 values were 80 and 30 nM for HeLa and MDAMB-231, respectively. The IC50 for MCF-7 was notably higher at ˜110 nM, which may be due to the deficiency in the terminal executioner caspase 3 and reliance on other effector caspases for apoptosis (see, e.g. M. Burek, et al. Oncogene 2006, 25, 2213-2222; R. U. Janicke, et al. J. Biol. Chem., 1998, 273, 9357-9360). As expected, native MBP-APO and ND APO NC did not notably decrease the viability of any cell lines tested, consistent with the inability to enter cells and release MBP-APO in cytoplasm, respectively. The morphologies of MDA-MB-231 and HFF cells were examined under various treatments. Only the S—S APO NC treated MDA-MB-231 cells exhibited blebbing and shrinkage, hallmarks of apoptotic cell death (FIG. 9e and FIG. 14). Using TUNEL assay, S—S APO NC treated MDA-MB-231 also showed nuclear fragmentation associated with apoptosis (green fluorescence from

Fluor 488), whereas all other samples, native MBP-APO and ND APO NC at the same concentration (FIG. 14), as well as HFF treated with 200 nM S—S APO NC (FIG. 9e), had no sign of apoptosis. Collectively, these results demonstrated that the released MBP-APO upon degradation in cytoplasm retains the potency and selectivity as the transgenically expressed apoptin in previous studies (see e.g., C. Backendorf, et al. Annu. Rev. Pharmacol. Toxicol. 2008, 48, 143-169).

Having demonstrated that S—S APO NC is highly effective in killing various tumor cell lines in vitro, we further examined its potency in mouse xenograft model. Female athymic nude (nu/nu) mice were subcutaneously grafted on the back flank with 5×10⁶ MCF-7 breast cancer cells. When the tumor volume reached 100-200 mm³ (day 0), mice were randomly separated into different groups and treated with intratumoral injection of PBS, MBP-APO, S—S APO NC. In addition, S—S NC with bovine serum albumin (S—S BSA NC) was added as a nonlethal protein cargo control testing the effects of the S—S NC polymer component on tumor cells in vivo. Tumors treated with saline, S—S BSA NC or native MBP-APO expanded rapidly and reached the maximum limit (>2500 mm³) within 12 days. In sharp contrast, tumor growth was significantly delayed when treated with S—S APO NC. Fixed tumor tissues collected from each treatment group was examined for DNA fragmentation using in situ TUNEL assay. The images revealed the highest level of cell apoptosis for the tumor harvested from mice treated with S—S

APO NC, correlating well with the significantly delayed tumor growth observed for this treatment group and confirming that tumor growth inhibition was indeed due to apoptin-mediated apoptosis. Collectively, the xenograft study verified that the degradable nanocapsule effectively delivered recombinant MBPAPO proteins to tumor cells in vivo, which was highly effective in limiting tumor progression.

In conclusion, we were able to deliver the high molecular weight complex of the tumor-selective MBP-APO using a redox-responsive polymeric nanocapsule in vitro and in vivo. The choice and design of the nanocapsule is well-suited for diverse protein targets because of its mild preparation conditions, completely reversible encapsulation and efficient cell membrane penetration/release of the protein cargo in the cytoplasm. Our application here further illustrates how intracellular protein delivery using nanoscale system can provide new possibilities for achieving selective cancer therapy.

This concludes the description of the illustrative embodiments of the present invention. The foregoing description of one or more embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

1. A composition of matter comprising:
at least one polypeptide; and
a polymeric network, wherein:
the polymeric network is coupled together by disulfide bonds so as to form a shell that encapsulates the polypeptide and the disulfide bonds are disposed within the polymeric network in an orientation so that:
they are reduced when exposed to an intracellular environment; and
reduction of the disulfide bonds alters the shell in a manner that allows the polypeptide to migrate from the shell into the intracellular environment.

2. The composition of claim 1, wherein the polymeric network exhibits a surface charge of between 3 and 5 millivolts.

3. The composition of claim 1, wherein the shell is spherical and has a diameter of less than 150, 125, 100, 75, 50, 25, 20, 15, 10 or 5 nanometers.

4. The composition of claim 1, wherein the polypeptide is not coupled to the polymeric network.

5. The composition of claim 1, wherein the polypeptide comprises a native protein.

6. The composition of claim 1, wherein the polypeptide comprises a protein that induces cellular death.

7. The composition of claim 1, wherein the polypeptide comprises a detectable marker.

8. A method of delivering a polypeptide into an intracellular environment of a cell comprising of the steps of:
(a) combining the cell with a composition of matter comprising the polypeptide disposed within a polymeric network, wherein the polymeric network is crosslinked by disulfide bonds so as to form a shell that encapsulates the polypeptide;
(b) allowing the composition of (a) to cross a membrane of the cell and enter an intracellular environment of the cell; and
(3) allowing reduction of the disulfides bonds of the polymeric network so as alter the shell in a manner that allows the polypeptide to migrate from the shell into the intracellular environment;
so that the polypeptide is delivered into the intracellular environment of the cell.

9. The method of claim 8, wherein the cell is a human cell.

10. The method of claim 9, wherein the cell is a cancer cell.

11. The method of claim 9, wherein the polypeptide is selected for an ability to alter a metabolic pathway of the cell.

12. The method of claim 11, wherein the polypeptide induces cellular death.

13. The method of claim 12, wherein the polypeptide is apoptin.

14. A method of forming a polymeric nanocapsule comprising the steps of:
(a) forming a mixture comprising:
a polypeptide,
a plurality of polymerizable monomers; and
a crosslinking agent selected for its ability to form disulfide bonds;
(b) allowing the plurality of polymerizable monomers and the crosslinking agent to adsorb to surfaces of the a polypeptide;
(c) initiating polymerization of the plurality of polymerizable monomers and the crosslinking agent at interfaces between the monomers and the polypeptide:
so that the polymeric nanocapsule is formed, wherein the polymeric nanocapsule encapsulates the polypeptide.

15. The method of claim 14, wherein the plurality of polymerizable monomers comprises an acrylamide.

16. The method of claim 14, wherein the crosslinking agent comprises a cystamine moiety.

17. The method of claim 14, wherein polymerization is initiated by adding a free radical initiator to the mixture.

18. The method of claim 14, wherein the polypeptide is selected for an ability to alter the transcription of a gene within a human cell.

19. The method of claim 14, wherein the mixture comprises a plurality of polypeptides associated within a protein complex.

20. The method of claim 15, wherein the polypeptide is not coupled to the polymeric nanocapsule following the polymerization of the plurality of polymerizable monomers and the crosslinking agent.
